(19) United States
(12) Patent Application Publication
(10) Pub. No.: US 2010/0075500 A1
(43) Pub. Date: Mar. 25, 2010

(54) Metal polishing slurry and chemical mechanical polishing method

(75) Inventors: Masaru Yoshikawa, Kanagawa (JP); Tadashi Inaba, Kanagawa (JP); Hiroshi Inada, Shizuoka (JP); Takamitsu Tomiga, Shizuoka (JP)

Correspondence Address: AKERMAN SENTERFITT, 8100 BOONE BOULEVARD, SUITE 700, VIENNA, VA, 22182-2683, US

(73) Assignee: FUJIFILM Corporation, Tokyo (JP)

(21) Appl. No.: 12/382,461

(22) Filed: Mar. 17, 2009

(30) Foreign Application Priority Data
Sep. 22, 2008 (JP) 2008-243245

(51) Int. Cl. H01L 21/463; H01L 21/465; C09K 13/00; C07D 249/04; C07D 249/08

(52) U.S. Cl. 438/692; 252/79.1; 548/255; 548/262.2; 257/E21.484; 257/E21.485

(57) ABSTRACT

The invention provides a metal polishing slurry containing a compound represented by the general formula (1): (X¹)ₙ-L wherein X¹ represents a heterocycle containing at least one nitrogen atom, n represents an integer of 2 or more, and L represents a linking group having a valence of 2 or more, provided that X¹s whose number is n may be the same or different, an oxidizer and an organic acid; and a method of chemical mechanical polishing using such slurry. The metal polishing slurry and the chemical mechanical polishing method are used in chemical mechanical polishing in the step of manufacturing semiconductor devices and enable a high polishing rate to be achieved while causing minimal dishing in polishing an object (wafer).

This application claims priority on Japanese patent application No. 2008-243245 which was filed on Sep. 22, 2008, the entire contents of which are hereby incorporated by reference. In addition, the entire contents of the literatures cited in this specification are also hereby incorporated by reference.

### BACKGROUND OF THE INVENTION

This invention relates to a novel compound, a metal polishing slurry for use in chemical mechanical planarization in the step of manufacturing semiconductor devices, and a polishing method using such metal polishing slurry.

In the development of semiconductor devices typified by semiconductor integrated circuits (hereinafter referred to as “LSI devices”), the trend toward smaller sizes and higher processing speeds has created a need in recent years for higher density and higher integration by the adoption of miniaturization and multilayer constructions of interconnection. Various techniques are being used to this end, including chemical mechanical polishing (hereinafter referred to as “CMP”).

CMP is an essential technique for carrying out, for example, the surface planarization of a film to be processed (e.g., an interlayer dielectric film), plug formation, and buried metal interconnect formation, and this technology is used to carry out substrate planarization and to remove surplus metal thin film during the formation of interconnections (see, for example, U.S. Pat. No. 4,944,836 and JP 2-278822 A).

CMP generally involves attaching a polishing pad onto a circular platen, impregnating the surface of the polishing pad with a polishing slurry, pressing the front side of a substrate (wafer) against the pad, and rotating both the platen and the substrate while applying a predetermined pressure (polishing pressure) from the back side of the substrate so as to planarize the front side of the substrate by the mechanical friction that arises.

The metal polishing slurry used in CMP typically includes an abrasive (such as alumina or silica) and an oxidizer (such as hydrogen peroxide). It is believed that polishing takes place with oxidization of the metal surface by the oxidizer and removal of the resulting oxide film by the abrasive. The procedure is described, for example, in Journal of Electrochemical Society, 1991, vol. 138, No. 11, pages 3460 to 3464.

However, CMP conducted by using such metal polishing slurry containing a solid abrasive is associated with the risk of scratches formed by the polishing (scratches), excessive polishing of the entire polishing surface (thinning), deformation of the polished metal surface in the shape of a dish (dishing), and excessive polishing of the insulator between metal interconnections and dish-shape deformation of the wiring metal surface (erosion). For example, JP 8-64594 A and JP 8-83780 A describe that inclusion of 1,2,3-benzotriazole and 2-aminothiazole in the polishing slurry is an effective means for suppressing such defects.

However, the inventors of the present invention have made a study on the dishing phenomenon and found that the dishing phenomenon cannot be sufficiently improved by the use of 1,2,3-benzotriazole and 2-aminothiazole.

High-speed polishing cannot be performed while reducing dishing. The present invention has been made in view of this problem and aims at achieving the following objects.

### SUMMARY OF THE INVENTION

Accordingly, an object of the present invention is to provide a metal polishing slurry which is capable of achieving a high polishing rate while reducing dishing in the polishing of an object to be polished (wafer). Another object of the present invention is to provide a chemical mechanical polishing method using such metal polishing slurry.

The inventors of the present invention have made an intensive study to solve the foregoing problem and as a result found that this problem can be solved by the metal polishing slurry and the polishing method using such metal polishing slurry as described below. The invention has been thus completed.

The metal polishing slurry and the polishing method using the same as well as the compound that may be advantageously used according to the present invention are as follows:
(1) A metal polishing slurry for use in chemical mechanical polishing in a step of manufacturing semiconductor devices, comprising a compound represented by the general formula (1):

(X¹)ₙ-L General formula (1)

wherein X¹ represents a group having a heterocycle which contains at least one nitrogen atom, n represents an integer of 2 or more, and L represents a linking group having a valence of two or more, provided that X¹s whose number is n may be the same or different; an oxidizer; and an organic acid.
(2) The metal polishing slurry according to (1) above, wherein the linking group represented by L in the general formula (1) contains at least one group selected from the group consisting of ureido group, thioureido group, amide group, ester group, carbonate group, carbamate group, sulfonamide group, sulfonureido group, hydroxy group, ether group, thioether group, amino group, carboxy group, sulfo group and heterocyclic group.
(3) The metal polishing slurry according to (1) or (2) above, wherein the linking group represented by L in the general formula (1) contains at least one heterocycle.
(4) The metal polishing slurry according to (3) above, wherein the heterocycle contained in the linking group represented by L in the general formula (1) is 1,2,4-triazole, benzotriazole or tetrazole.
(5) The metal polishing slurry according to any one of (1) to (4) above, wherein the heterocycle represented by X¹ in the general formula (1) is tetrazole, 1,2,4-triazole, 1,2,3-triazole or benzotriazole.
(6) The metal polishing slurry according to any one of (1) to (5) above, wherein the metal polishing slurry further contains a surfactant represented by the general formula (2):

R—Ar—O—Ar—SO₃⁻M⁺ General formula (2)

wherein R represents a linear or branched alkyl group having 8 to 20 carbon atoms, Ar represents an aryl group, and M⁺ represents hydrogen ion, an alkali metal ion or ammonium.
(7) The metal polishing slurry according to any one of (1) to (6) above further containing colloidal silica.
(8) The metal polishing slurry according to (7) above, wherein the colloidal silica has a primary particle size of 20 to 40 nm and an average association degree of up to 2.
(9) The metal polishing slurry according to (7) or (8) above, wherein the colloidal silica is one in which at least some of surface silicon atoms are modified with aluminum atoms.
(10) The metal polishing slurry according to any one of (1) to (9) above, wherein the organic acid is an amino acid.
(11) A method of chemical mechanical polishing comprising the steps of: rotating a polishing platen on which a polishing pad is disposed; feeding the metal polishing slurry of any one of (1) to (10) above to the polishing pad; and polishing an object while moving the polishing pad and the object to be polished relative to each other with a surface of the object in contact with the polishing pad.
(12) A compound represented by the following general formula (3):

(X²)ₙ-L General formula (3)

(wherein X² represents tetrazole, 1,2,4-triazole, 1,2,3-triazole or benzotriazole, L represents a linking group having a valence of 2 or more which contains at least one group selected from the group consisting of ureido group, thioureido group, amide group, ester group, sulfonamide group, sulfonureido group, hydroxy group, carbamate group, ether group, amino group, carboxy group, sulfo group and heterocyclic group, n is an inter of 2 or more; provided that X²s whose number is n may be the same or different), or a passivation film-forming agent which comprises this compound and is used for a chemical mechanical polishing slurry in a step of manufacturing semiconductor devices.
(13) The compound according to (12) above or the passivation film-forming agent which comprises this compound and is used for a chemical mechanical polishing slurry in the step of manufacturing semiconductor devices, wherein, in the general formula (3), X² represents tetrazole or 1,2,3-triazole, and the linking group having a valence of 2 or more as represented by L contains at least one group selected from the group consisting of ureido group, amide group, hydroxy group, carbamate group, ether group, and amino group.
(14) The compound according to (12) above, wherein, in the general formula (3), n is 2 to 6, X² is tetrazole, and L contains at least one group selected from the group consisting of ureido group, amide group and carbamate group.

The chemical mechanical polishing method of the present invention can increase the polishing rate while minimizing the dishing phenomenon on the substrate surface.

The metal polishing slurry of the present invention enables a high polishing rate to be achieved while causing minimal dishing in polishing an object (wafer).

The compound of the present invention can produce a metal polishing slurry which enables a high polishing rate to be achieved while causing minimal dishing in polishing an object (wafer).

### DETAILED DESCRIPTION OF THE PREFERRED EMBODIMENT

Specific embodiments of the present invention are described below.

The compound of the present invention is described below.

The compound of the present invention is represented by the general formula (3):

(X²)ₙ-L General formula (3)

wherein X² represents tetrazole, 1,2,4-triazole, 1,2,3-triazole or benzotriazole, L represents a linking group having a valence of 2 or more which contains at least one group selected from the group consisting of ureido group, thioureido group, amide group, ester group, sulfonamide group, sulfonureido group, hydroxy group, carbamate group, ether group, amino group, carboxy group, sulfo group and heterocyclic group, provided that X²s whose number is n may be the same or different. The compound of the present invention is useful as an additive of a chemical mechanical polishing slurry in the step of manufacturing semiconductor devices as will be described later. The compound of the present invention is particularly useful as a passivation film-forming agent in CMP.

In the general formula (3), X² represents tetrazole, 1,2,4-triazole, 1,2,3-triazole or benzotriazole, and X²s whose number is n may be the same or different.

In the general formula (3), L represents a linking group having a valence of 2 or more which contains at least one group selected from the group consisting of ureido group, thioureido group, amide group, ester group, sulfonamide group, sulfonureido group, hydroxy group, carbamate group, ether group, amino group, carboxy group, sulfo group and heterocyclic group, and n is preferably an integer of 2 to 6.

The linking group may contain two or more of these groups.

In the present specification, the linking group L is used in the sense of the group capable of connecting together X²s whose number is n.

The linking group may contain a hydrocarbon group in addition to at least one group selected from the group consisting of ureido group, thioureido group, amide group, ester group, sulfonamide group, sulfonureido group, hydroxy group, ether group, amino group, carboxy group, sulfo group and heterocyclic group.

The hydrocarbon group that may be contained in the linking group is not particularly limited. Examples of the hydrocarbon group include linear or branched alkylene groups such as dimethylene group, trimethylene group, tetramethylene group, hexamethylene group, 1,1,3-trimethylhexylene group; alicyclic hydrocarbon groups such as cyclohexylene group; aromatic hydrocarbon groups such as phenylene group, tolylene group and xylylene group; and heterocycles.

The hydrocarbon group may have heteroatoms such as oxygen atom, nitrogen atom and sulfur atom in the form of, for example, an ether bond, a sulfide bond, a polysulfide bond such as —S—S, a secondary amine, or a tertiary amine.

The hydrocarbon groups may be used alone or in combination of two or more to form the linking group.

In a preferred embodiment, the linking group has a hydrocarbon group whose both ends each independently have a ureido group, thioureido group, amide group, ester group, sulfonamide group, sulfonureido group, or ether group, and the ureido group, thioureido group, amide group, ester group, sulfonamide group or sulfonureido group is attached to X¹ or X².

In such a case, the ureido group becomes a ureylene group, the thioureido group becomes a thioureylene group, the amide group becomes —NHCO—, the sulfonamide group becomes —SO₂NH—, and the sulfonureido group becomes a sulfonureylene group.

The linking group may have two hydrocarbon groups bonded together through an ether group, ureido group, thioureido group, amide group, ester group, sulfonamide group, sulfonureido group, or ether group.

In a preferred embodiment, a hydroxy group, amino group, carboxy group, sulfo group, ureido group, thioureido group, amide group, ester group, sulfonamide group, or sulfonureido group is attached to the hydrocarbon group in the linking group.

If possible, L in the general formula (3) may further have a substituent.

Examples of the substituent that can be introduced include halogen atom (fluorine atom, chlorine atom, bromine atom, or iodine atom), alkyl group (which may be a linear, branched, or cyclic alkyl group including polycyclic alkyl group such as bicycloalkyl group, and which may contain active methine group), alkenyl group, alkynyl group, aryl group, heterocyclic group (which is not limited by the position of substitution), acyl group, alkoxycarbonyl group, aryloxycarbonyl group, heterocyclic oxycarbonyl group, carbamoyl group (which is optionally substituted as in the case of N-hydroxycarbamoyl group, N-acylcarbamoyl group, N-sulfonylcarbamoyl group, N-carbamoylcarbamoyl group, thiocarbamoyl group, or N-sulfamoylcarbamoyl group), carbazoyl group, carboxy group or its salt, oxalyl group, oxamoyl group, cyano group, carbonimidoyl group, formyl group, hydroxy group, alkoxy group (including a group containing ethyleneoxy group or propyleneoxy group as its repeating unit), aryloxy group, heterocyclic oxy group, acyloxy group, (alkoxy or aryloxy) carbonyloxy group, carbamoyloxy group, sulfonyloxy group, amino group, (alkyl, aryl, or heterocyclic) amino group, acylamino group, sulfonamide group, ureido group, thioureido group, N-hydroxyureido group, imide group, (alkoxy or aryloxy) carbonylamino group, sulfamoylamino group, semicarbazide group, thiosemicarbazide group, hydrazino group, ammonio group, oxamoylamino group, N-(alkyl or aryl) sulfonylureido group, N-acylureido group, N-acylsulfamoylamino group, hydroxyamino group, nitro group, heterocyclic group containing quaternized nitrogen atom (for example, pyridinio group, imidazolio group, quinolinio group, or isoquinolinio group), isocyano group, imino group, mercapto group, (alkyl, aryl, or heterocyclic) thio group, (alkyl, aryl, or heterocyclic) dithio group, (alkyl or aryl) sulfonyl group, (alkyl or aryl) sulfinyl group, sulfo group, sulfamoyl group (which is optionally substituted as in the case of N-acylsulfamoyl group or N-sulfonylsulfamoyl group), phosphino group, phosphinyl group, phosphinyloxy group, phosphinylamino group, and silyl group.

From the viewpoint that the metal polishing slurry used in chemical mechanical polishing in the step of manufacturing semiconductor devices can suppress dishing of the substrate surface while increasing the polishing rate, preferable examples of the linking group having a valence of 2 or more as represented by L in the general formula (3) include a linking group containing any of ureido group, thioureido group, amide group, ester group, sulfonamide group, hydroxy group, carbamate group, ether group, amino group, carboxy group, sulfo group and heterocyclic group, and a divalent to hexavalent linking group having a substituted hydroxy, carboxy or sulfo group.

The linking group having a valence of 2 or more as represented by L in the general formula (3) is more preferably a linking group containing at least one group selected from the group consisting of ureido group, amide group, carbamate group, ether group, and amino group, or a linking group having a substituted carbamate or hydroxy group.

The linking group having a valence of 2 or more as represented by L in the general formula (3) is even more preferably a linking group having at least one heterocycle. It is preferable for the linking group having a valence of 2 or more as represented by L to contain a heterocycle because a higher polishing rate and reduced dishing are possible.

If the substitution position is not limited, illustrative examples of the heterocycle contained in the linking group having a valence of 2 or more as represented by L in the general formula (3) include monovalent groups corresponding to pyridine, pyrazine, pyridazine, pyrimidine, triazine, quinoline, isoquinoline, quinazoline, cinnoline, phthalazine, quinoxaline, pyrrole, indole, furan, benzofuran, thiophene, benzothiophene, pyrazole, imidazole, benzimidazole, triazole, tetrazole, oxazole, benzoxazole, thiazole, benzothiazole, isothiazole, benzisothiazole, thiadiazole, isoxazole, benzisoxazole, pyrrolidine, piperidine, piperazine, imidazolidine, and thiazoline. Of these, 1,2,4-triazole, benzotriazole, and tetrazole are preferred and 1,2,4-triazole and tetrazole are more preferred.

In the compounds of the general formula (3), combinations in which n is 2 to 6, X² is tetrazole, 1,2,4-triazole or 1,2,3-triazole, and L is ureido group, amide group or carbamate group are preferred. In the compounds of the general formula (3), combinations in which n is 2, X² is tetrazole, L is ureido group or amide group are more preferred and combinations in which X² is tetrazole and L is ureido group are even more preferred. Combinations in which n is 2 to 6, X² is tetrazole, and L is ureido group, amide group or carbamate group are also preferred.

Specific examples of the linking group having a valence of 2 or more as represented by L in the general formula (3) are illustrated below.

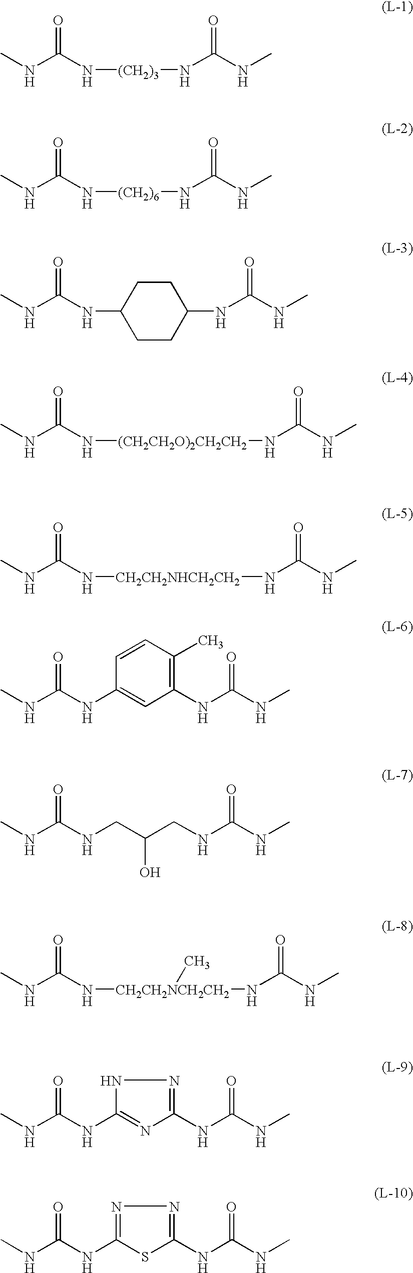

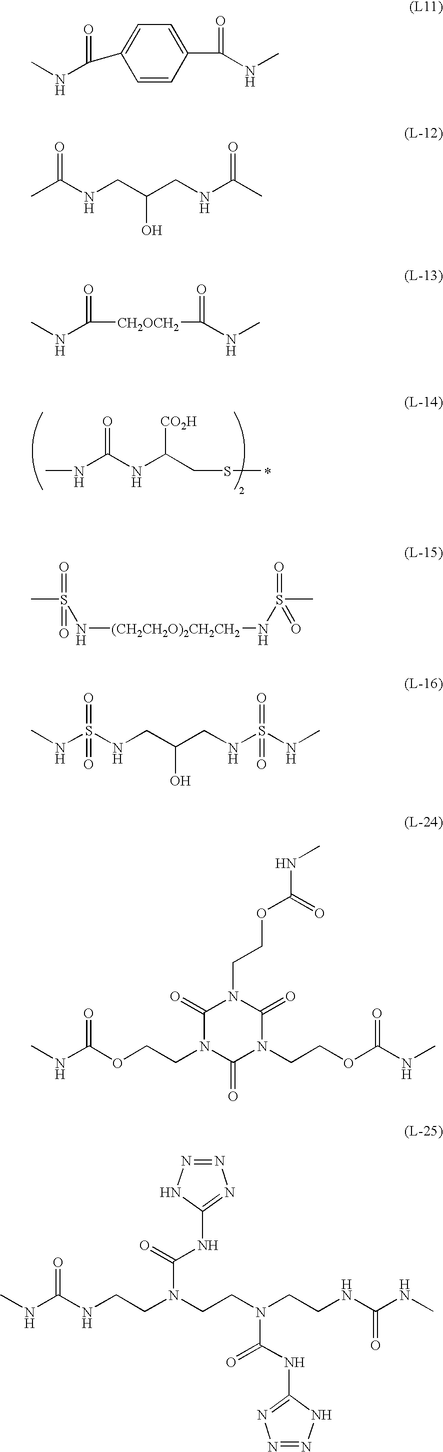

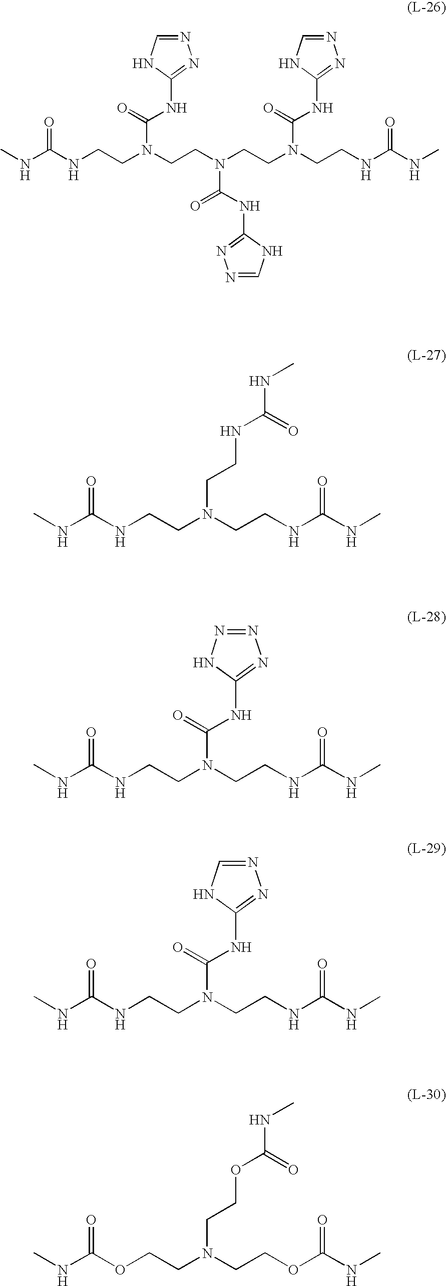

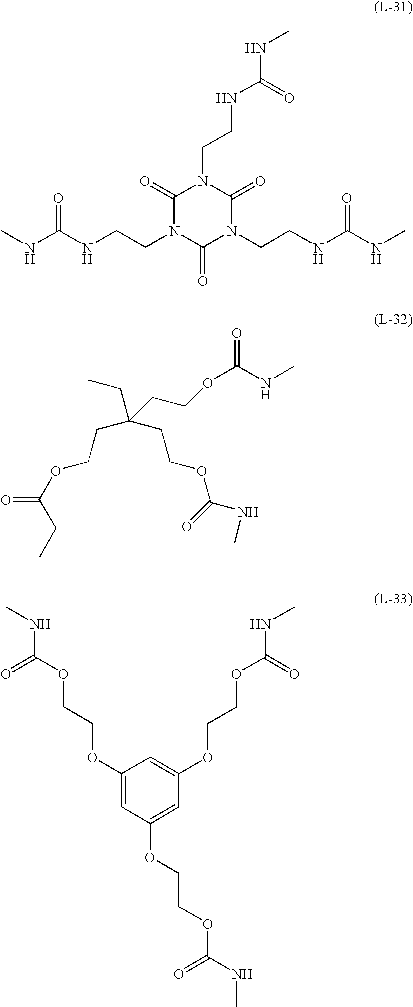

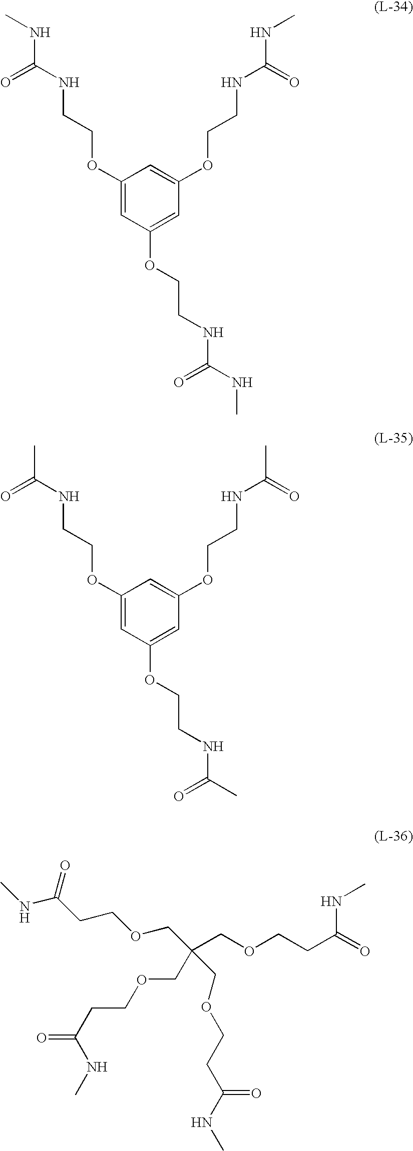

From the viewpoint that the metal polishing slurry used in chemical mechanical polishing in the step of manufacturing semiconductor devices can suppress dishing of the substrate surface while increasing the polishing rate, the compound represented by the general formula (3) is preferably one in which X² represents tetrazole or 1,2,3-triazole, and the divalent linking group represented by L contains at least one group selected from the group consisting of ureido group, amide group, hydroxy group, ether group and amino group.

Specific examples of the compound represented by the general formula (3) of the present invention are illustrated below.

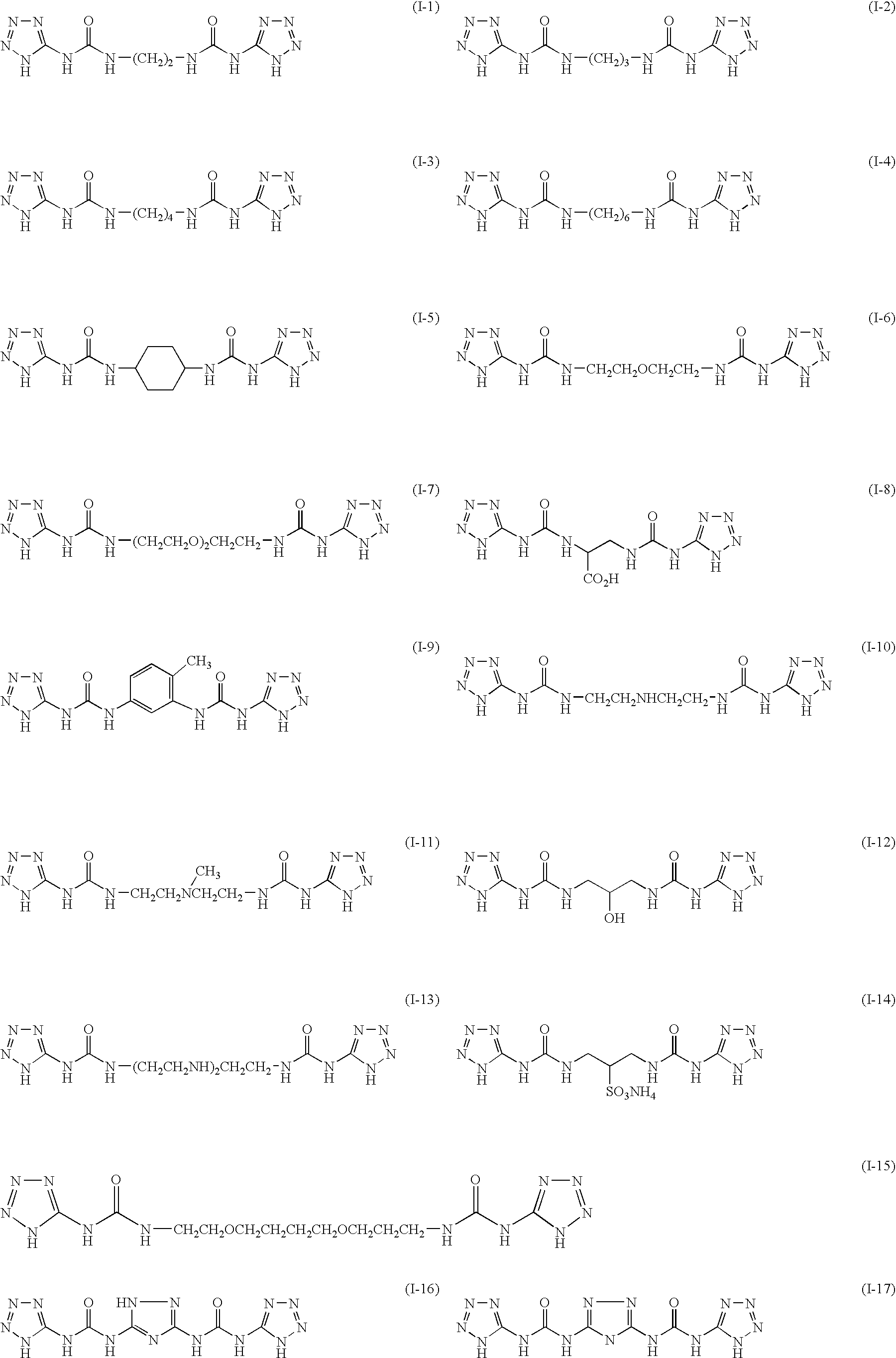

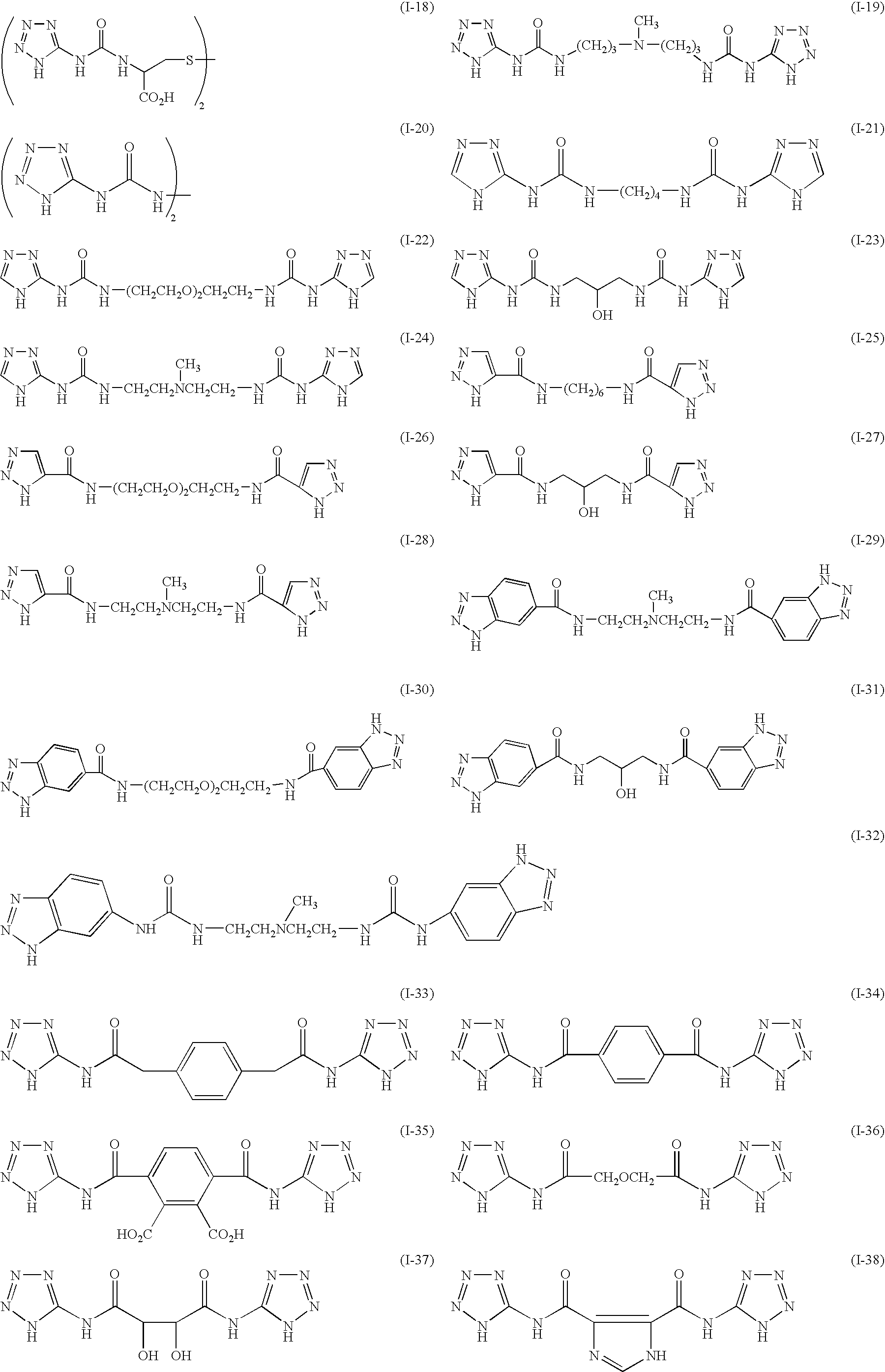

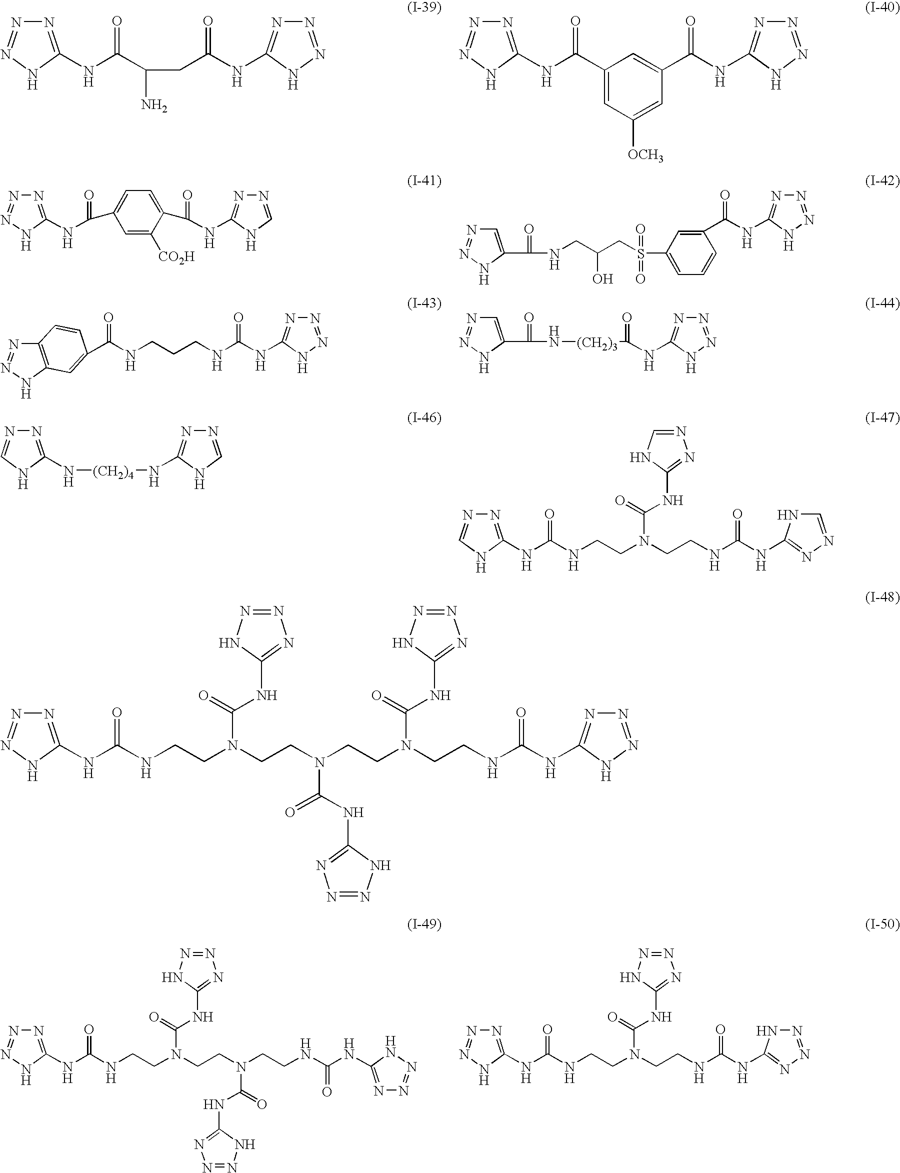

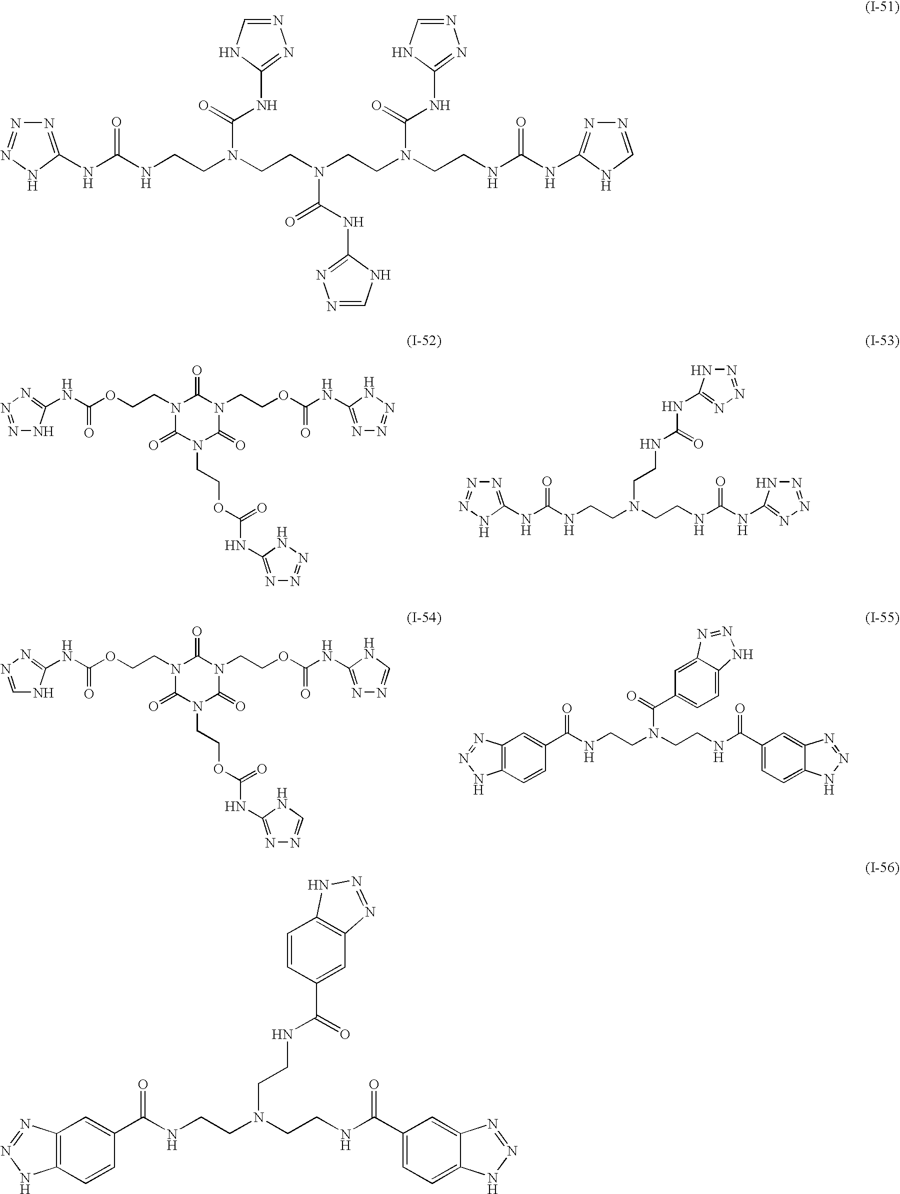

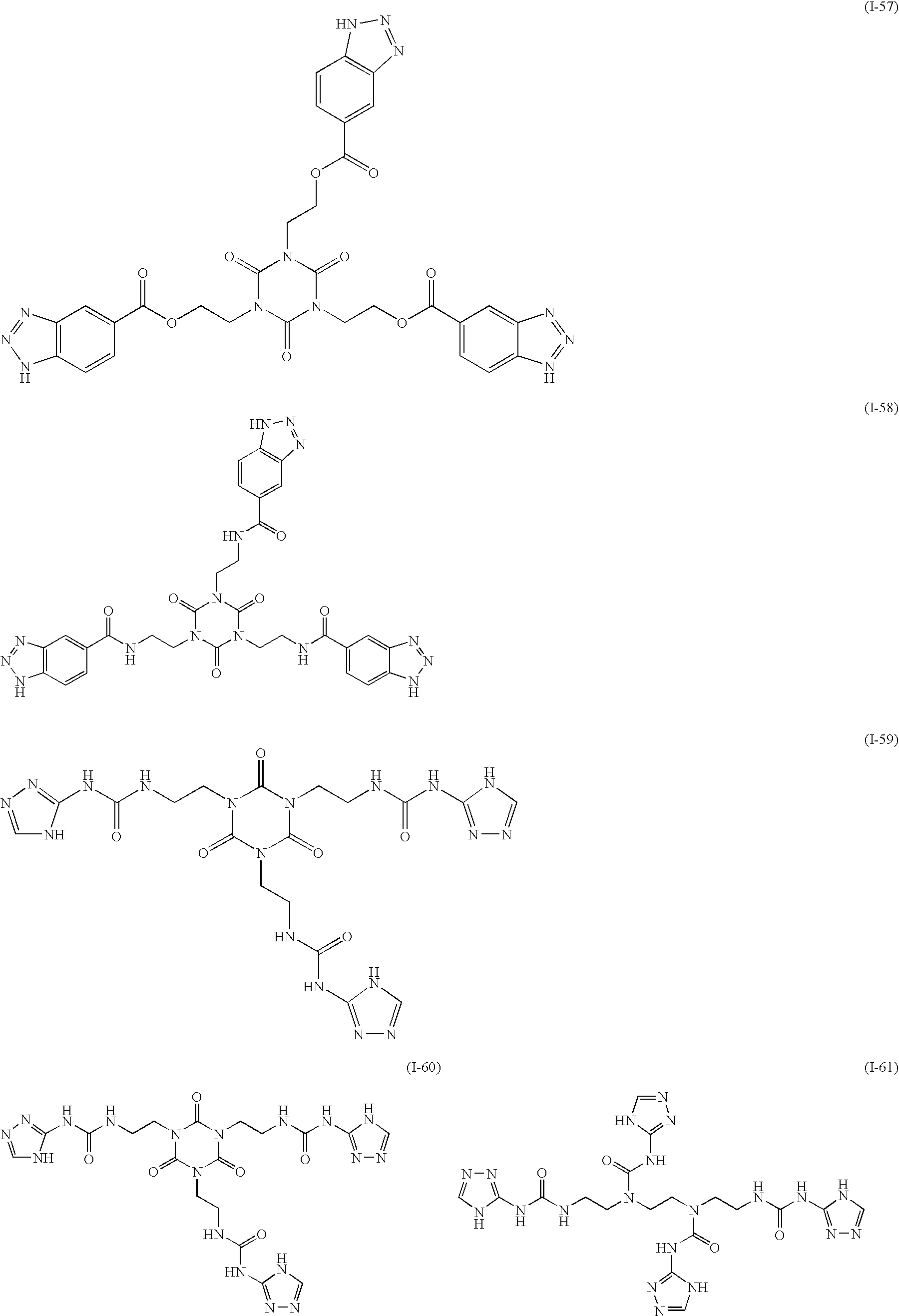

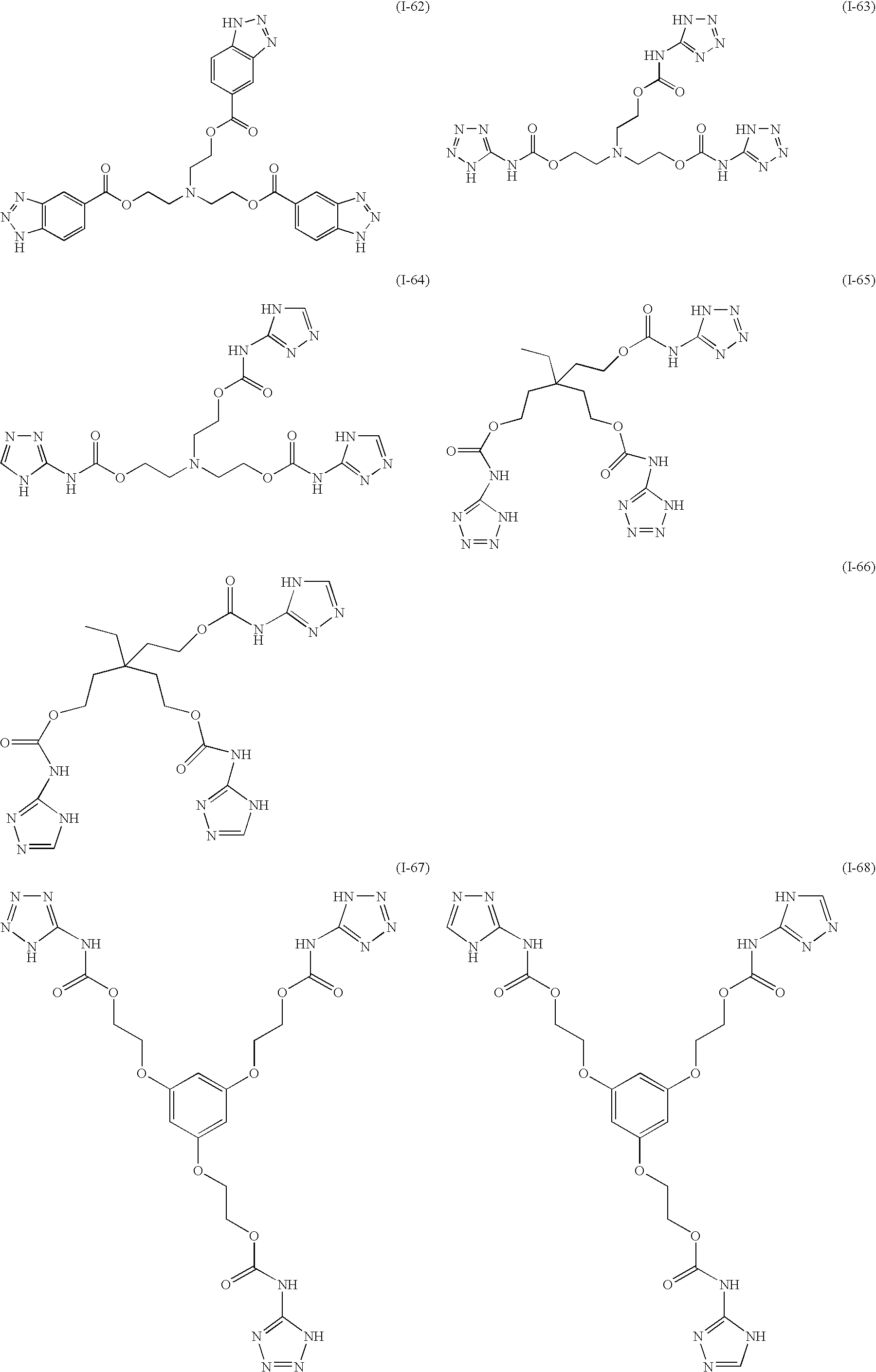

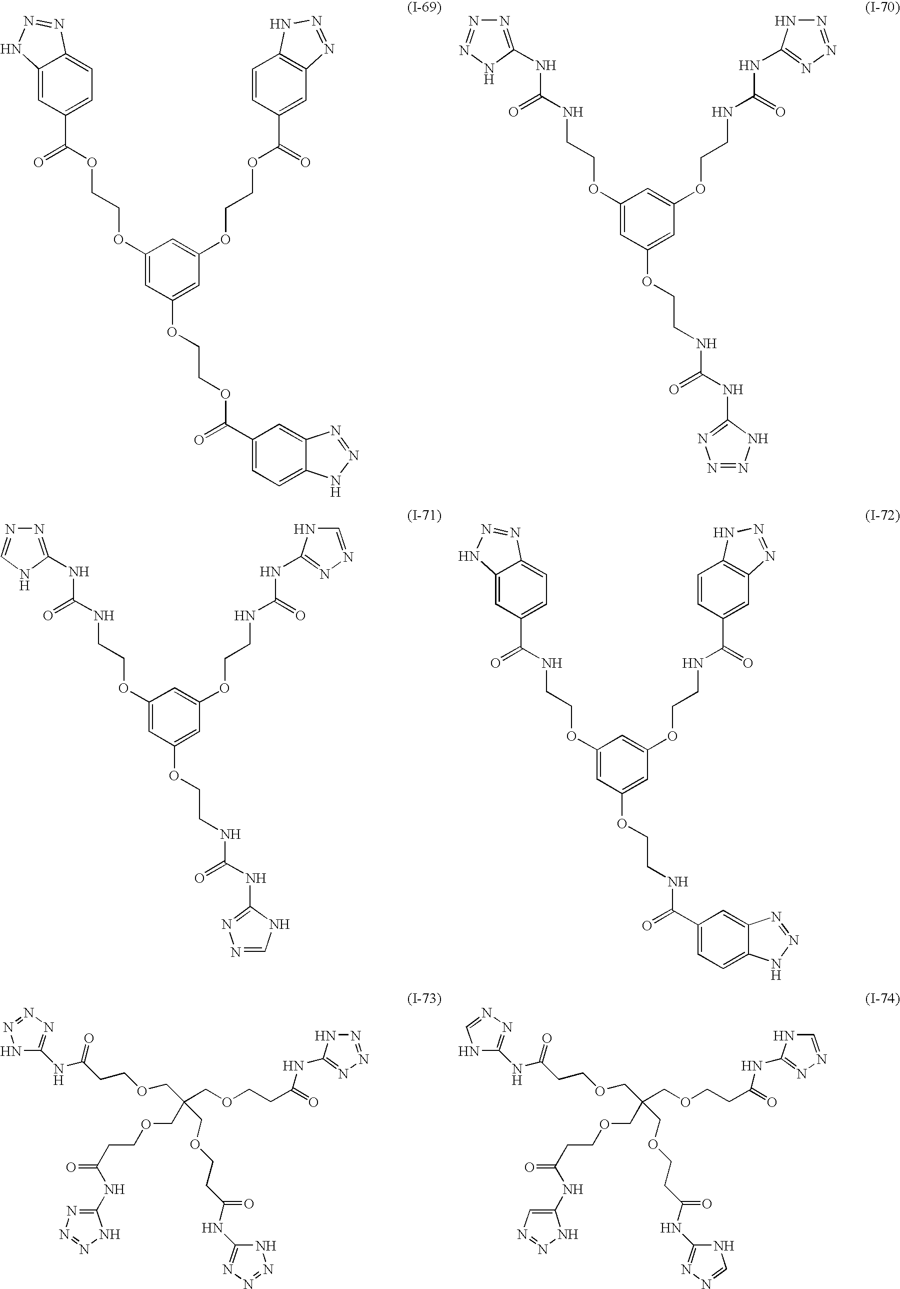

An exemplary method of producing the compound represented by the general formula (3) of the present invention includes producing the compound in which n is 2 through the reaction represented by the formula (4):

X³-A¹+B¹—R¹—B²+A²-X⁴→X³-L-X⁴ Formula (4)

(wherein X³ and X⁴ each represent tetrazole, 1,2,4-triazole, 1,2,3-triazole, or benzotriazole, R¹ represents a single bond, a hydrocarbon group, a heterocyclic group or a combination thereof, A¹ and A² are functional groups capable of each independently reacting with B¹ and B² to form a ureido group, a thioureido group, an amide group, an ester group, a sulfonamide group, a sulfonureido group, an ether group, an amino group or a sulfo group, L represents a divalent linking group containing at least one group selected from the group consisting of ureido group, thioureido group, amide group, ester group, sulfonamide group, sulfonureido group, hydroxy group, ether group, amino group, carboxy group, sulfo group and heterocyclic group, provided that X³ and X⁴ may be the same or different, A¹ and A² may be the same or different, and B¹ and B² may be the same or different. Even in the case where n is 3 or more, a compound in which 3 or more Bs are attached to R¹ may be used and reacted with three or more compounds such as X³-A¹ to produce the compound represented by the general formula (3) in the same manner.

The hydrocarbon group is defined as above.

A¹ and A² represent, for example, amino group, carboxy group, thiocarboxy group, hydroxy group, sulfo group, —NH—CO—O—R, —NH—CS—O—R, —NH—CO—NH—NH₂, —NH—CO—NH—OH, —CO—O—R, —CO—O—CO—R, —CO—Cl, —CO—NH—C—O—R, —N═C═O, —N═C═S, —OCN, —SCN, or —O—CO—OR.

B¹ and B² represent, for example, amino group, carboxy group, thiocarboxy group, hydroxy group, sulfo group, —NH—CO—O—R, —NH—CS—O—R, —NH—CO—NH—NH₂, —NH—CO—NH—OH, —CO—O—R, —CO—O—CO—R, —CO—O—SO₂—R, —CO—Cl, —CO—NH—CO—O—R, —N═C═O, —N═C═S, —OCN, —SCN, or —O—CO—OR.

R attached to —NH—CO—O—, —NH—CS—, —CO—O—, —CO—NH—CO—O—, or —O—CO—O is a hydrocarbon group, and the hydrocarbon group R is not particularly limited. Examples of the hydrocarbon group include alkyl groups such as methyl group, ethyl group and n-butyl group; and aromatic groups such as phenyl group.

Exemplary combinations of A¹ and A² with B¹ and B² include ones in which A¹ and A² are each amino group and B¹ and B² are each —NH—CO—O—R, —N═C═O, —CO—O—R, —CO—O—CO—R, or —CO—Cl; and ones in which A¹ and A² are —NH—CO—O—R and B¹ and B² are each amino group or hydroxy group.

Exemplary compounds represented by B¹—R¹—B² are illustrated below.

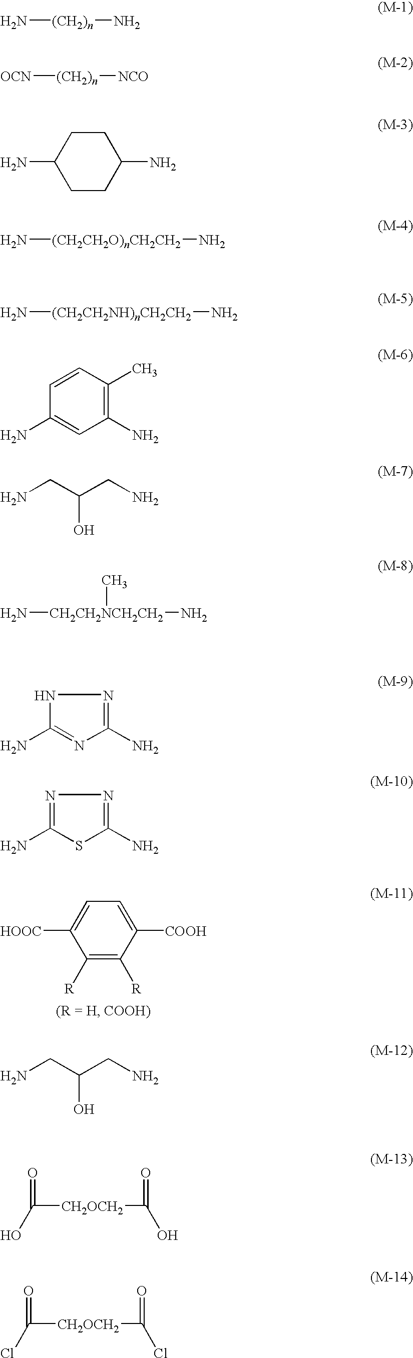

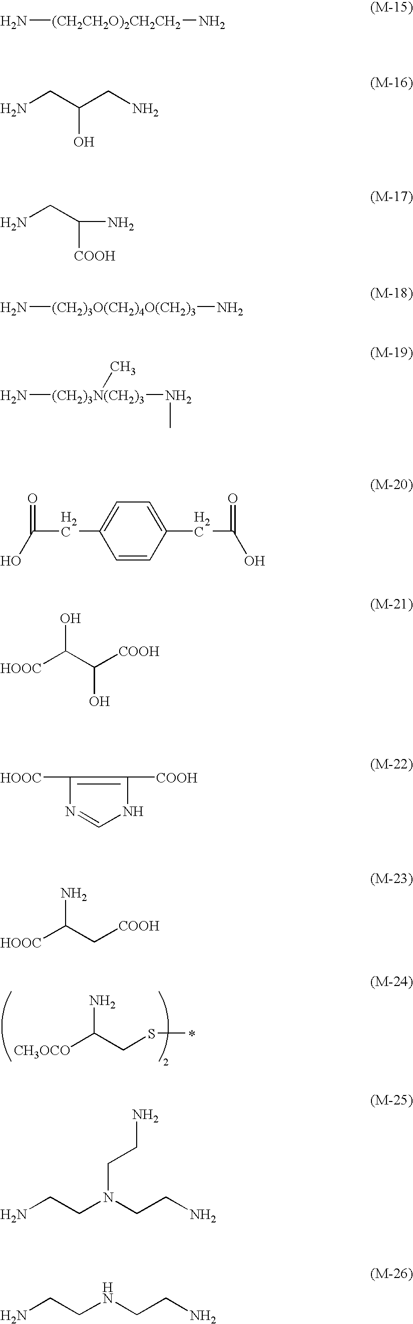

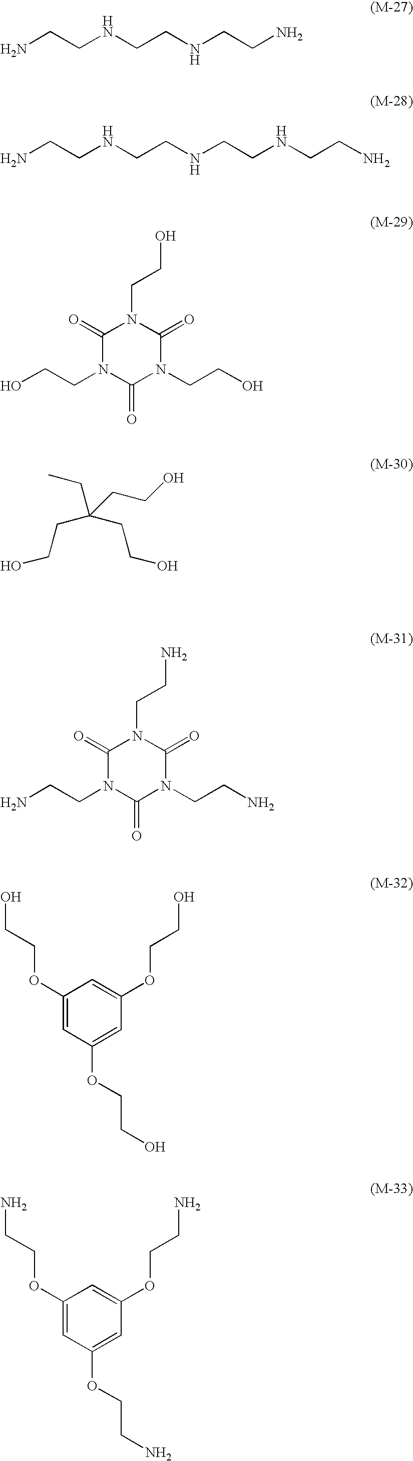

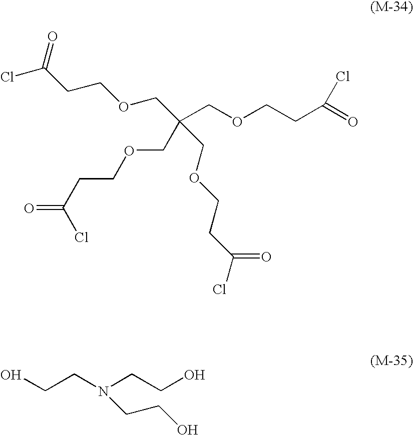

In M-1, n is an integer of 1 to 12.

In M-2, n is an integer of 1 to 12.

In M-4, n is an integer of 1 to 12.

In M-5, n is an integer of 1 to 12.

In M-18, n is an integer of 1 to 12.

M-1 to M-16 correspond to L-1 to L-16, respectively, and can be used to produce L-1 to L-16.

M-17 can be used to produce I-8. In the same manner, M-18, M-19, M-20, M-21, M-22, M-23 and M-24 can be used to produce I-15, I-19, I-33, I-37, I-38, I-39, and I-18, respectively.

M-25 can be used to produce I-53 and I-56, M-26 can be used to produce I-47, I-50 and I-55, and M-27 can be used to produce I-49 and I-61.

M-28 can be used to produce I-48 and I-51.

M-29 can be used to produce I-52, I-54 and I-57.

M-30 can be used to produce I-65 and I-66.

M-31 can be used to produce I-58, I-59 and I-60.

M-32 can be used to produce I-67, I-68 and I-69.

M-33 can be used to produce I-70, I-71 and I-72.

M-34 can be used to produce I-73 and I-74.

M-35 can be used to produce I-62, I-63 and I-64.

Exemplary compounds represented by X³-A¹ and A²-X⁴ are illustrated below.

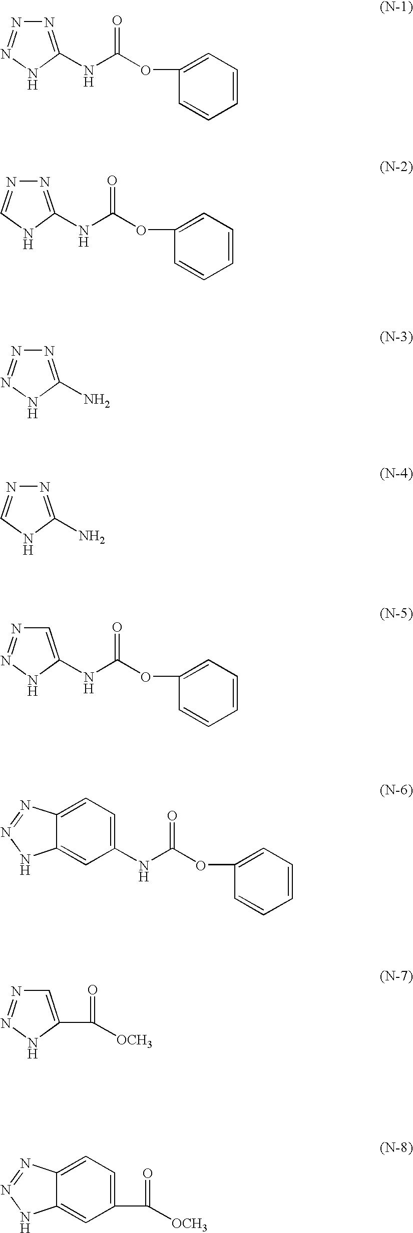

An exemplary method of reacting X³-A¹ and A²-X⁴ with B¹—R¹—B² include a method in which X³-A¹, A²-X⁴ and B¹—R¹—B² are used in such amounts that the number of moles of A¹ and A² is equivalent to the number of moles of B¹ and B², and reacted in a solvent such as acetonitrile or N-methylpyrrolidone under the condition of 0 to 100° C.

Catalysts such as acids (including Lewis acids) and bases (including Lewis bases) and condensing agents such as dicyclohexylcarbodiimide may be used for the reaction of X³-A¹ and A²-X⁴ with B¹—R¹—B².

The reaction of X³-A¹ and A²-X⁴ with B¹—R¹—B² may be followed by, for example, a hydrolysis reaction for substituent conversion.

As for its application, the compound represented by the general formula (3) of the present invention may be employed, for example, in a metal polishing slurry used in chemical mechanical polishing in the step of manufacturing semiconductor devices.

By incorporating the compound of the present invention in the metal polishing slurry used in chemical mechanical polishing in the step of manufacturing semiconductor devices, a high polishing rate and low dishing can be achieved at a time in polishing an object (wafer) with the metal polishing slurry.

### [Metal Polishing Slurry]

The metal polishing slurry of the present invention is described below.

The metal polishing slurry of the present invention is a metal polishing slurry characterized in that it is used in chemical mechanical polishing in the step of manufacturing semiconductor devices and contains a compound represented by the general formula (1):

(X¹)ₙ-L General formula (1)

(wherein X¹ represents a heterocycle containing at least one nitrogen atom, n represents an integer of 2 or more, and L represents a linking group having a valence of 2 or more, provided that X¹s whose number is n may be the same or different), an oxidizer, and an organic acid.

The metal polishing slurry of the present invention is used in chemical mechanical polishing in the step of manufacturing semiconductor devices, and the respective components are described below in detail. A single substance or a combination of two or more substances may be used for each component.

### [Compound Represented by the General Formula (1)]

The metal polishing slurry of the present invention contains the compound represented by the general formula (1).

Examples of the nitrogen-containing heterocycle represented by X¹ in the general formula (1) include pyrrole ring, pyrane ring, imidazole ring, pyrazole ring, thiazole ring, isothiazole ring, oxazole ring, isoxazole ring, pyridine ring, pyrazine ring, pyrimidine ring, pyridazine ring, pyrrolidine ring, pyrazolidine ring, imidazolidine ring, isoxazolidine ring, isothiazolidine ring, piperidine ring, piperazine ring, morpholine ring, thiomorpholine ring, indoline ring, isoindoline ring, pyrindine ring, indolizine ring, indole ring, indazole ring, purine ring, quinolizine ring, isoquinoline ring, quinoline ring, naphthyridine ring, phthalazine ring, quinoxaline ring, quinazoline ring, cinnoline ring, pteridine ring, acridine ring, perimidine ring, phenanthroline ring, carbazole ring, carboline ring, phenazine ring, anthyridine ring, thiadiazole ring, oxadiazole ring, triazine ring, 1,2,3-triazole ring, 1,2,4-triazole ring, tetrazole ring, benzimidazole ring, benzoxazole ring, benzothiazole ring, benzothiadiazole ring, benzofuroxan ring, naphthoimidazole ring, benzotriazole ring, and tetraazaindene ring.

From the viewpoint that a high polishing rate and reduced dishing can be simultaneously achieved with higher efficiency in polishing an object (wafer), tetrazole ring, 1,2,4-triazole ring, 1,2,3-triazole ring, and benzotriazole ring are preferred, with tetrazole ring, 1,2,4-triazole ring, and 1,2,3-triazole ring being more preferred and tetrazole ring being even more preferred.

X¹s whose number is n may be the same or different. n is an inter of 2 or more and preferably 2 to 10. Within this range, the compound represented by the general formula (1) can be industrially handled with ease at a reasonable price when used for the metal polishing slurry.

Examples of the linking group having a valence of 2 or more as represented by L in the general formula (1) include hydrocarbon groups such as alkylene groups (e.g., methylene group, ethylene group, trimethylene group, tetramethylene group, hexamethylene group, 1,4-cyclohexylene group, 1,1,3-trimethylhexylene group), arylene groups (e.g., p-phenylene group, m-phenylene group, naphthalene group), heterocyclic groups (e.g., pyridine ring-linking group, triazine ring-linking group, triazole ring-linking group, thiadiazole ring-linking group); and substituents such as ureido group, amide group, ester group, carbonate group, carbamate group, sulfonamide group, thioureido group, ether group, thioether group and amino group.

The linking group may be one having a valence of 2 or more in which two or more groups selected from those described above are linked to each other.

In a preferred embodiment, the linking group contains a hydrocarbon group in addition to the substituents such as ureido group, amide group, ester group, carbonate group, carbamate group, sulfonamide group, thioureido group, ether group, thioether group and amino group.

If possible, L in the general formula (1) may further have a substituent.

Examples of the substituent that can be introduced include halogen atom (fluorine atom, chlorine atom, bromine atom, or iodine atom), alkyl group (which may be a linear, branched, or cyclic alkyl group including polycyclic alkyl group such as bicycloalkyl group, and which may contain active methine group), alkenyl group, alkynyl group, aryl group, heterocyclic group (which is not limited by the position of substitution), acyl group, alkoxycarbonyl group, aryloxycarbonyl group, heterocyclic oxycarbonyl group, carbamoyl group (which is optionally substituted as in the case of N-hydroxycarbamoyl group, N-acylcarbamoyl group, N-sulfonylcarbamoyl group, N-carbamoylcarbamoyl group, thiocarbamoyl group, or N-sulfamoylcarbamoyl group), carbazoyl group, carboxy group or its salt, oxalyl group, oxamoyl group, cyano group, carbonimidoyl group, formyl group, hydroxy group, alkoxy group (including a group containing ethyleneoxy group or propyleneoxy group as its repeating unit), aryloxy group, heterocyclic oxy group, acyloxy group, (alkoxy or aryloxy) carbonyloxy group, carbamoyloxy group, sulfonyloxy group, amino group, (alkyl, aryl, or heterocyclic) amino group, acylamino group, sulfonamide group, ureido group, thioureido group, N-hydroxyureido group, imide group, (alkoxy or aryloxy) carbonylamino group, sulfamoylamino group, semicarbazide group, thiosemicarbazide group, hydrazino group, ammonio group, oxamoylamino group, N-(alkyl or aryl) sulfonylureido group, N-acylureido group, N-acylsulfamoylamino group, hydroxyamino group, nitro group, heterocyclic group containing quaternized nitrogen atom (for example, pyridinio group, imidazolio group, quinolinio group, or isoquinolinio group), isocyano group, imino group, mercapto group, (alkyl, aryl, or heterocyclic) thio group, (alkyl, aryl, or heterocyclic) dithio group, (alkyl or aryl) sulfonyl group, (alkyl or aryl) sulfinyl group, sulfo group, sulfamoyl group (which is optionally substituted as in the case of N-acylsulfamoyl group or N-sulfonylsulfamoyl group), phosphino group, phosphinyl group, phosphinyloxy group, phosphinylamino group, and silyl group.

From the viewpoint that a high polishing rate and reduced dishing can be simultaneously achieved with higher efficiency in polishing an object (wafer), preferable examples of the linking group having a valence of 2 or more as represented by L in the general formula (1) include a linking group containing any of ureido group, amide group, ester group, carbonate group, carbamate group, sulfonamide group, hydroxy group, ether group, thioether group, amino group, carboxy group, sulfo group and heterocyclic group, and a linking group having a valence of 2 or more which has a substituted hydroxy, carboxy or sulfo group.

The divalent linking group represented by L in the general formula (1) is more preferably a linking group containing at least one group selected from the group consisting of ureido group, amide group, ether group, and amino group, or a linking group having a substituted hydroxy group.

The linking group having a valence of 2 or more as represented by L in the general formula (1) is even more preferably a linking group having at least one heterocycle.

If the substitution position is not limited, illustrative examples of the heterocycle contained in the linking group having a valence of 2 or more as represented by L in the general formula (1) include monovalent groups corresponding to pyridine, pyrazine, pyridazine, pyrimidine, triazine, quinoline, isoquinoline, quinazoline, cinnoline, phthalazine, quinoxaline, pyrrole, indole, furan, benzofuran, thiophene, benzothiophene, pyrazole, imidazole, benzimidazole, triazole, tetrazole, oxazole, benzoxazole, thiazole, benzothiazole, isothiazole, benzisothiazole, thiadiazole, isoxazole, benzisoxasole, pyrrolidine, piperidine, piperazine, imidazolidine, and thiazoline. Of these, 1,2,4-triazole, benzotriazole, and tetrazole are preferred and 1,2,4-triazole and tetrazole are more preferred.

Specific examples of the linking group having a valence of 2 or more as represented by L in the general formula (1) are illustrated below. The following examples exclude the groups which have already been illustrated in connection with the general formula (3).

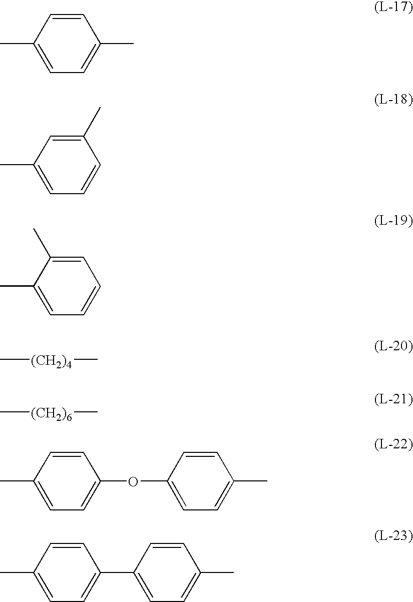

Specific examples of the compound which is contained in the metal polishing slurry of the present invention and is represented by the general formula (1) are illustrated below. However, the present invention is not limited thereto. The following examples exclude the compounds which have already been illustrated in connection with the general formula (3).

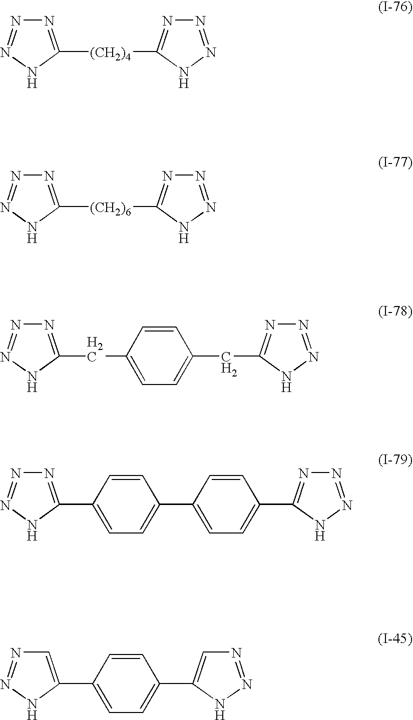

From the viewpoint that a high polishing rate and reduced dishing can be simultaneously achieved with higher efficiency in polishing an object (wafer), the compound represented by the general formula (1) is preferably one represented by the general formula (3).

The compound represented by the general formula (1) is not particularly limited for its production method. An exemplary method includes a method of producing the compound represented by the general formula (3) of the present invention.

The compound represented by the general formula (1) that may be used in the present invention is preferably added in a total amount of 1×10⁻⁸ to 1×10⁻¹ mol, more preferably 1×10⁻⁷ to 1×10⁻² mol, and even more preferably 1×10⁻⁶ to 1×10⁻³ mol, per liter of the metal polishing slurry at the time of use in polishing.

### (Oxidizer)

The metal polishing slurry of the present invention contains an oxidizer. In the practice of the invention, the oxidizer is not particularly limited as long as it is a compound capable of oxidizing a metal to be polished.

Illustrative examples of the oxidizer include hydrogen peroxide, peroxides, nitrates, iodates, periodates, hypochlorites, chlorites, chlorates, perchlorates, persulfates, bichromates, permanganates, ozonated water, silver (II) salts, and iron (III) salts.

Of these, hydrogen peroxide is preferable in terms of a high polishing rate and excellent resistance to dishing.

The amount of oxidizer added is preferably from 0.003 to 8 mol, more preferably from 0.03 to 6 mol, and most preferably from 0.1 to 4 mol, per liter of the metal polishing slurry at the time of use in polishing. In other words, the amount of oxidizer added is preferably at least 0.003 mol/L to ensure sufficient metal oxidation and a high CMP rate, but is preferably not more than 8 mol/L to prevent roughening of the polished surface.

### (Organic Acid)

The metal polishing slurry of the present invention contains an organic acid. The organic acid as used herein refers to a compound which has a different structure from the oxidizer for use in metal oxidation.

The organic acid is desirably water-insoluble and examples thereof include amino acids and other acids. Of these, amino acids are preferably used in terms of the high water solubility.

The amino acid is more suitably selected from, for example, the following group including glycine, L-alanine, β-alanine, N-methylglycine, L-2-aminobutyric acid, L-norvaline, L-valine, L-leucine, L-norleucine, L-isoleucine, L-alloisoleucine, L-phenylalanine, L-proline, L-ornithine, L-lysine, taurine, L-serine, L-threonine, L-allothreonine, L-homoserine, L-tyrosine, 3,5-diiodo-L-tyrosine, dihydroxyethylglycine, β-(3,4-dihydroxyphenyl)-L-alanine, L-thyroxine, 4-hydroxy-L-proline, L-cysteine, L-methionine, L-ethionine, L-lanthionine, L-cystathionine, L-cystine, L-cysteic acid, L-aspartic acid, L-glutamic acid, S-(carboxymethyl)-L-cysteine, 4-aminobutyric acid, L-asparagine, L-glutamine, azaserine, L-arginine, L-canavanine, L-citrulline, δ-hydroxy-L-lysine, creatine, L-kynurenine, L-histidine, 1-methyl-L-histidine, 3-methyl-L-histidine, ergothioneine, L-tryptophan, actinomycin C1, apamin, angiotensin I, angiotensin II, and antipain.

The organic acid other then the amino acids is suitably selected from, for example, the following group including formic acid, acetic acid, propionic acid, butyric acid, valeric acid, 2-methylbutyric acid, n-hexanoic acid, 3,3-dimethylbutyric acid, 2-ethylbutyric acid, 4-methylpentanoic acid, n-heptanoic acid, 2-methylhexanoic acid, n-octanoic acid, 2-ethylhexanoic acid, benzoic acid, glycolic acid, salicylic acid, glyceric acid, oxalic acid, malonic acid, succinic acid, glutaric acid, adipic acid, pimelic acid, maleic acid, phthalic acid, malic acid, tartaric acid, citric acid, lactic acid, hydroxyethyliminodiacetic acid, iminodiacetic acid, and ammonium salts or alkali metal salts of these acids.

Of these, glycine, N-methylglycine, dihydroxyethylglycine, malic acid, tartaric acid, citric acid, hydroxyethyliminodiacetic acid, and iminodiacetic acid are preferable in terms of effectively suppressing the etching rate while maintaining practically acceptable CMP rate.

The amount of organic acid added is preferably from 0.0005 to 0.5 mol, more preferably from 0.005 to 0.3 mol, and most preferably from 0.01 to 0.1 mol, per liter of the polishing slurry at the time of use in polishing. That is, to suppress etching, the amount of acid added is preferably not more than 0.5 mol/L, but is preferably at least 0.0005 mol/L to achieve a sufficient effect.

### (Abrasive)

The metal polishing slurry of the present invention preferably further contains an abrasive in terms of the excellent polishing effect.

Examples of the abrasive that may be preferably used include silicas (e.g. precipitated silica, fumed silica, colloidal silica, and synthetic silica), ceria, alumina, titania, zirconia, germania, manganese oxide, silicon carbide, polystyrene, polyacryl, and polyterephthalate.

Use of colloidal silica is particularly preferable because significant effects of the present invention including a high polishing rate and low dishing can be simultaneously achieved with higher efficiency in polishing an object (wafer).

The abrasive preferably has an average particle size of 5 to 200 nm, and use of an abrasive with an average particle size of 20 to 70 nm is particularly preferable because the effects of the present invention are fully achieved.

A colloidal silica having a primary particle size of 20 to 40 nm and an average association degree of 2 or less is advantageously used as the abrasive that may be incorporated in the metal polishing slurry of the present invention. Incorporation of such colloidal silica in the metal polishing slurry enables a high polishing rate and minimal dishing to be simultaneously achieved with higher efficiency in polishing an object (wafer) and is therefore preferable.

The colloidal silica more preferably has a primary particle size of 20 nm to 30 nm from the viewpoint that a high polishing rate can be more efficiently achieved while causing minimal dishing in polishing an object (wafer).

The primary particle size of the particles used in the present invention is an average particle diameter calculated from the particle size distribution obtained by a dynamic light scattering method.

The association degree refers to a value obtained by dividing the diameter of secondary particles produced by aggregation of primary particles by the diameter of the primary particles (the diameter of the secondary particles/the diameter of the primary particles). Accordingly, when the abrasive has an association degree of 1, the abrasive solely comprises monodispersed primary particles.

The secondary particle size can be measured with an electron microscope or other instrument.

The colloidal silica that may be incorporated in the metal polishing slurry of the present invention is preferably a colloidal silica in which at least some of surface silicon atoms are modified with aluminum atoms (this colloidal silica is hereinafter sometimes referred to as “specific colloidal silica”). By using such colloidal silica having at least some of its surface silicon atoms modified with aluminum atoms, dishing can be further reduced.

An exemplary method that may be advantageously used to obtain the specific colloidal silica involves adding an aluminate compound such as ammonium aluminate to a dispersion containing a colloidal silica. More specifically, examples thereof include a method in which a silica sol obtained by addition of an aqueous alkali aluminate solution is heated at 80 to 250° C. for 0.5 to 20 hours and brought into contact with a cation exchange resin and optionally an anion exchange resin; a method in which an acidic silicate solution and an aqueous aluminum compound solution are added to an SiO₂-containing aqueous alkali solution or aqueous alkali metal hydroxide solution, a method in which an aluminum compound-containing acidic silicate solution is added to an SiO₂-containing aqueous alkali solution or aqueous alkali metal hydroxide solution, and a method in which the thus prepared aluminum compound-containing alkaline silica sol is dealkalized by treatment with a cation exchange resin. These methods are described in detail in JP 3463328 B and JP 63-123807 A, and their disclosure can be applied to the present invention.

The abrasive is preferably added in an amount of 0.05 to 20 g per liter of the metal polishing slurry used, and incorporation of the abrasive in an amount of 0.2 to 5 g enables the effects of the invention to be significantly achieved and is therefore particularly preferable.

In cases where the metal polishing slurry contains no abrasive or the abrasive concentration is less than 0.01% by weight, the polishing rate and resistance to dishing can be preferably improved by adjusting the pH to at least 3.5 and particularly at least 4.0.

The metal polishing slurry of the present invention may further contain a surfactant represented by the general formula (2):

R—Ar—O—Ar—SO₃⁻M⁺ General formula (2)

wherein R represents a linear or branched alkyl group having 8 to 20 carbon atoms, Ar represents an aryl group, and M⁺ represents hydrogen ion, an alkali metal ion or ammonium.

### [Surfactant Represented by the General Formula (2)]

The metal polishing slurry of the present invention preferably contains a surfactant represented by the general formula (2):

R—Ar—O—Ar—SO₃⁻M⁺ General formula (2)

in order to further reduce dishing.

In the general formula (2), R represents a linear or branched alkyl group having 8 to 20 carbon atoms.

The alkyl group preferably has 10 to 20 carbon atoms and more preferably 12 to 20 carbon atoms. The alkyl group represented by R may be a linear or branched alkyl group, and preferably a linear alkyl group.

Specific examples of the alkyl group represented by R include decyl group, undecyl group, dodecyl group, tridecyl group, tetradecyl group, pentadecyl group, hexadecyl group, heptadecyl group, octadecyl group, nonadecyl group and eicosyl group.

Of these, dodecy group, tridecyl group, tetradecyl group, pentadecyl group, octadecyl group, nonadecyl group and eicosyl group are preferred from the viewpoint that the dishing phenomenon can be further suppressed.

In the general formula (2), Ar represents an aryl group. Examples of the aryl group represented by Ar include phenyl group, naphthyl group, anthryl group, and phenanthryl group.

Of these, phenyl group is preferred from the viewpoint that the dishing phenomenon can be further suppressed.

The two Ar groups in the general formula (2) may be the same or different, and preferably the same.

The alkyl group or aryl group may be further substituted.

Examples of the substituent that can be introduced include halogen atom (fluorine atom, chlorine atom, bromine atom, or iodine atom), alkyl group (which may be a linear, branched, or cyclic alkyl group including polycyclic alkyl group such as bicycloalkyl group, and which may contain active methine group), alkenyl group, alkynyl group, aryl group, heterocyclic group (which is not limited by the position of substitution), acyl group, alkoxycarbonyl group, aryloxycarbonyl group, heterocyclic oxycarbonyl group, carbamoyl group (which is optionally substituted as in the case of N-hydroxycarbamoyl group, N-acylcarbamoyl group, N-sulfonylcarbamoyl group, N-carbamoylcarbamoyl group, thiocarbamoyl group, or N-sulfamoylcarbamoyl group), carbazoyl group, carboxy group or its salt, oxalyl group, oxamoyl group, cyano group, carbonimidoyl group, formyl group, hydroxy group, alkoxy group (including a group containing ethyleneoxy group or propyleneoxy group as its repeating unit), aryloxy group, heterocyclic oxy group, acyloxy group, (alkoxy or aryloxy) carbonyloxy group, carbamoyloxy group, sulfonyloxy group, amino group, (alkyl, aryl, or heterocyclic) amino group, acylamino group, sulfonamide group, ureido group, thioureido group, N-hydroxyureido group, imide group, (alkoxy or aryloxy) carbonylamino group, sulfamoylamino group, semicarbazide group, thiosemicarbazide group, hydrazino group, ammonio group, oxamoylamino group, N-(alkyl or aryl) sulfonylureido group, N-acylureido group, N-acylsulfamoylamino group, hydroxyamino group, nitro group, heterocyclic group containing quaternized nitrogen atom (for example, pyridinio group, imidazolio group, quinolinio group, or isoquinolinio group), isocyano group, imino group, mercapto group, (alkyl, aryl, or heterocyclic) thio group, (alkyl, aryl, or heterocyclic) dithio group, (alkyl or aryl) sulfonyl group, (alkyl or aryl) sulfinyl group, sulfo group, sulfamoyl group (which is optionally substituted as in the case of N-acylsulfamoyl group or N-sulfonylsulfamoyl group), phosphino group, phosphinyl group, phosphinyloxy group, phosphinylamino group, and silyl group.

Of these, an alkyl group and sulfo group are preferable in terms of the excellent scratch resistance.

In addition, M⁺ in the general formula (2) represents hydrogen ion, an alkali metal ion, or ammonium.

Sodium ion and potassium ion are preferable alkali metal ions represented by M⁺, with sodium ion being more preferable.

Ammonium (NH₄⁺) represented by M⁺ also includes ammonium having a hydrogen atom substituted with an alkyl group. Examples of the ammonium include methyl ammonium and ethyl ammonium.

M⁺ is more preferably hydrogen ion or ammonium, and most preferably hydrogen ion.

Exemplary surfactants represented by the general formula (2) include alkyl diphenyl ether disulfonic acids such as dodecyl diphenyl ether disulfonic acid, tetradecyl diphenyl ether disulfonic acid, hexadecyl diphenyl ether disulfonic acid, octadecyl diphenyl ether disulfonic acid, and eicosyl diphenyl ether disulfonic acid, and their salts; alkyl diphenyl ether monosulfonic acids such as dodecyl diphenyl ether monosulfonic acid, tetradecyl diphenyl ether monosulfonic acid, hexadecyl diphenyl ether monosulfonic acid, octadecyl monophenyl ether disulfonic acid, and eicosyl monophenyl ether disulfonic acid, and their salts; and dodecyl dinaphthyl ether disulfonic acid, dodecyl dianthryl ether disulfonic acid, dodecyl dinaphthyl ether monosulfonic acid, dodecyl dianthryl ether monosulfonic acid, and their salts.

The surfactant represented by the general formula (2) preferably contains an alkyl diphenyl ether disulfonic acid or its salt in terms of reducing the dishing. A mixture of an alkyl diphenyl ether disulfonic acid and an alkyl diphenyl ether monosulfonic acid or a mixture of their salts is preferable.

In the case of such mixture, the alkyl diphenyl ether monosulfonic acid is preferably incorporated in the mixture in an amount of at least 10% by mole, more preferably at least 30% by mole, and even more preferably at least 50% by mole.

The surfactant represented by the general formula (2) is preferably incorporated in an amount of 0.0001% by weight to 0.1% by weight, more preferably 0.0005% by weight to 0.05% by weight, and even more preferably 0.001% by weight to 0.01% by weight in relation to the metal polishing slurry used.

The method used for synthesizing the surfactant represented by the general formula (2) is not particularly limited, and commercially available surfactants may be preferably used.

Next, a surfactant and a hydrophilic polymer that may be used in combination with the surfactant represented by the general formula (2) in cases where the metal polishing slurry of the present invention further contains the surfactant represented by the general formula (2) is described.

In the present invention, various surfactants and hydrophilic polymers as described below may be used in combination.

Examples of the anionic surfactant include carboxylate salts, sulfonate salts, sulfate salts, and phosphate salts.

Examples of the cationic surfactant include aliphatic amine salts, aliphatic quaternary ammonium salts, benzalkonium chloride salts, benzethonium chloride, pyridinium salts, and imidazolinium salts.

Examples of the amphoteric surfactant include carboxybetaine surfactants, aminocarboxylate salts, imidazolinium betaines, lecithins, and alkylamine oxides.

Examples of the nonionic surfactant include ether surfactants, ether ester surfactants, ester surfactants, and nitrogen-containing surfactants.

Fluorosurfactants may also be used.

Examples of the hydrophilic polymer include polyglycols such as polyethylene glycol, polyvinyl alcohol, polyvinylpyrrolidone, polysaccharides such as alginic acid, and carboxylic acid-containing polymers such as polymethacrylic acid.

Of these, acids or ammonium salts thereof are desirable because there is no contamination by alkali metals, alkaline earth metals and halides.

Of the illustrated compounds, cyclohexanol, ammonium salt of polyacrylic acid, polyvinyl alcohol, succinic amide, polyvinylpyrrolidone, polyethylene glycol, and a polyoxyethylene/polyoxypropylene block polymer are more preferred in terms of the excellent scratch resistance and high polishing rate.

The weight-average molecular weight of these surfactants and hydrophilic polymers is preferably from 500 to 100,000, and most preferably from 2,000 to 50,000.

The surfactant other than represented by the general formula (2) and/or the hydrophilic polymer are preferably incorporated in an amount of 0.0001% by weight to 1.0% by weight, more preferably 0.0005% by weight to 0.5% by weight, and even more preferably 0.001% by weight to 0.1% by weight in relation to the metal polishing slurry used.

The metal polishing slurry of the present invention may further contain a heterocyclic compound other than the compound represented by the general formula (1).

Examples of the heterocyclic compound that may also be used in the present invention include, but are not limited to, 1,2,3,4-tetrazole, 5-amino-1,2,3,4-tetrazole, 5-methyl-1,2,3,4-tetrazole, 1,2,3-triazole, 4-amino-1,2,3-triazole, 4,5-diamino-1,2,3-triazole, 1,2,4-triazole, 3-amino-1,2,4-triazole, 3,5-diamino-1,2,4-triazole, and benzotriazole.

### [Other Components]

The metal polishing slurry of the present invention may also contain other components and examples thereof include additives such as a pH adjuster and a chelating agent.

### (pH Adjuster)

An acidic agent, an alkaline agent, or a buffering agent is preferably added to the metal polishing slurry of the present invention to adjust the pH to a predetermined value.

Examples of the acidic agent include inorganic acids such as sulfuric acid, nitric acid, boric acid, and phosphoric acid. Of these, sulfuric acid is preferred.

Examples of the alkaline agent and buffering agent include nonmetallic alkaline agents such as ammonia, ammonium hydroxide, organic ammonium hydroxides (e.g., tetramethylammonium hydroxide), and alkanolamines (e.g., diethanolamine, triethanolamine, and triisopropanol amine); alkali metal hydroxides such as sodium hydroxide, potassium hydroxide, and lithium hydroxide; carbonates such as sodium carbonate; phosphates such as trisodium phosphate; borates; tetraborates; and hydroxybenzoates.

Ammonium hydroxide, potassium hydroxide, lithium hydroxide, and tetramethylammonium hydroxide are particularly preferred alkaline agents.

The pH adjuster should be added in such an amount that the pH is maintained in a preferable range, and the content is preferably from 0.0001 to 1.0 mol, and more preferably from 0.003 to 0.5 mol, per liter of the metal polishing slurry at the time of use in polishing.

In terms of further suppressing the dishing phenomenon and also in terms of stability in the polishing rate and polishing slurry, the metal polishing slurry for use in polishing preferably has a pH of 3 to 12, more preferably 4 to 9, and most preferably 5 to 8. Use of the acidic agent, alkaline agent and buffering agent enables the pH of the metal polishing slurry of the present invention to be adjusted within the above-defined preferable range.

### (Chelating Agent)

The metal polishing slurry according to the present invention may optionally contain a chelating agent (i.e. water softener) to reduce an adverse effect of polyvalent metal ion contaminants.

Examples of the chelating agent that may be used include general-purpose water softeners (agents for preventing precipitation of calcium and magnesium) and their analogous compounds. The chelating agents may be optionally used in combination of two or more.

The chelating agent may be used in a sufficient amount to sequester the contaminants including metal ions such as polyvalent metal ions. For example, the chelating agent is preferably added in an amount of 0.0003 to 0.07 mol per liter of the metal polishing slurry actually used for polishing.

The metal polishing slurry of the present invention is not particularly limited for its production. For example, the metal polishing slurry can be produced by mixing together a compound represented by the general formula (1), an oxidizer and an organic acid, as well as optionally used abrasive, surfactant represented by the general formula (2), surfactant and hydrophilic polymer that may be used with the surfactant represented by the general formula (2), heterocyclic compound other than the compound represented by the general formula (1), additives and water.

A component (A) which contains an oxidizer and a component (B) which contains a compound represented by the general formula (1) and an organic acid, as well as optionally used abrasive, surfactant represented by the general formula (2), surfactant and hydrophilic polymer that may be used with the surfactant represented by the general formula (2), heterocyclic compound other than the compound represented by the general formula (1), additives and water may be separately produced to obtain the metal polishing slurry of the present invention.

Unless otherwise specified, the term “metal polishing slurry” as used herein encompasses not only polishing slurries having a composition (concentration) suitable to use in polishing but also polishing concentrates optionally diluted before use. Before use in polishing, the concentrates are diluted with water or an aqueous solution to generally 1 to 20 times its original volume.

### [Method of Chemical Mechanical Polishing]

The chemical mechanical polishing method of the present invention is a polishing method which involves feeding the metal polishing slurry of the present invention to the polishing pad on the polishing platen, rotating the polishing platen and polishing an object while moving the polishing pad and the object to be polished relative to each other with the polishing pad in contact with the object surface.

The chemical mechanical polishing method of the present invention is described below in detail.

### (Polishing Apparatus)

An apparatus with which the polishing method of the present invention can be implemented is first described.

The polishing apparatus applicable to the present invention may be an ordinary polisher having a holder which holds the object to be polished (such as semiconductor substrate) having a surface to be polished and a polishing platen (provided with a motor whose number of revolution is variable or the like) onto which a polishing pad is attached. Use may be made of, for example, FREX300 (manufactured by Ebara Corporation).

### (Polishing Pressure)

In the polishing method of the present invention, polishing is preferably carried out at a polishing pressure in terms of contact pressure between the surface to be polished and the polishing pad of 3,000 to 25,000 Pa and more preferably 6,500 to 14,000 Pa.

### (Rotation Speed of Polishing Platen)

In the polishing method of the prevent invention, polishing is preferably carried out by rotating the polishing platen at 50 to 200 rpm and more preferably at 60 to 150 rpm.

### (Method of Feeding Polishing Slurry)

In the prevent invention, the metal polishing slurry is continuously fed to the polishing pad on the polishing platen with a pump during polishing of a metal of interest. While the amount of polishing slurry fed to the polishing pad is not limited, it is preferable that the surface of the polishing pad be steadily covered with the metal polishing slurry.

The metal polishing slurry that may be used in the polishing method of the invention is not particularly limited as long as it is the metal polishing slurry of the present invention.

The polishing method of the present invention may also use a metal polishing slurry obtained by diluting a concentrated metal polishing slurry with water or an aqueous solution. Methods for diluting a concentrated slurry with water or an aqueous solution include a method in which a line that feeds a concentrated metal polishing slurry and a line that feeds water or an aqueous solution are joined together at same intermediate point so that the respective fluids may be mixed, with the resulting metal polishing slurry dilution being fed to the polishing pad. Mixing of the concentrated slurry and the water or aqueous solution may be carried out by conventional methods including a method that involves causing the two fluids to run under pressure through narrow passages so that the fluids may collide and mix with each other; a method in which a material such as glass tubing is packed in a pipe so as to make the flow of liquid split and confluent repeatedly; or a method that provides blades within a pipe which are powered to rotate.

Another exemplary dilution method that may be used in the present invention includes a process which independently provides a line that feeds a metal polishing slurry and a line that feeds water or an aqueous solution, feeds predetermined amounts of fluid from the lines to the polishing pad, and carries out mixing of the two fluids by moving the polishing pad and the surface to be polished relative to each other.

In another process which may be applied in the present invention, predetermined amounts of concentrated metal polishing slurry and of water or an aqueous solution are added to a single vessel and mixed, and the slurry diluted to a predetermined concentration is fed to the polishing pad to carry out polishing.

In addition to these methods, the present invention may also use a method in which the ingredients to be contained in the metal polishing slurry are separated into at least two components, which are diluted with water or an aqueous solution before use and fed to the polishing pad. In this case, it is preferable for the component containing an oxidizer and the component containing a compound represented by the general formula (1) and an organic acid to be separately fed as the metal polishing slurry.

More specifically, it is preferable that the component (A) contain an oxidizer and the component (B) contain a compound represented by the general formula (1) and an organic acid, as well as optionally used abrasive, surfactant represented by the general formula (2), surfactant and hydrophilic polymer that may be used with the surfactant represented by the general formula (2), heterocyclic compound other than the compound represented by the general formula (1), additives and water.

In a preferred embodiment, the components (A) and (B) are diluted with water or an aqueous solution before use. In this case, three lines for feeding the component (A), component (B) and water or aqueous solution, respectively, are necessary. The three lines may be coupled to a single line so that mixing is realized in the latter line. Alternatively, two of the three lines may be joined together before joining the third line where the components are mixed. For example, the component containing difficult-to-dissolve additives and the other component are initially mixed in a long passage so as to ensure a sufficient dissolution time, following which water or an aqueous solution is added from its feeding line joined downstream of the mixing passage.

The three lines may be each directly brought to the polishing pad so that the components are mixed together by the movement of the polishing pad relative to the surface to be polished and the mixed components are fed. Alternatively, the three components may be mixed together in a vessel before feeding the mixture to the polishing pad. It is also possible to prepare the metal polishing slurry as a concentrate and feed it to the surface to be polished separately from the diluting water.

### (Amount of Polishing Slurry Fed)

In the polishing method of the present invention, the metal polishing slurry is preferably fed onto the polishing platen in an amount of 50 to 500 ml/min and more preferably 100 to 300 ml/min.

### (Polishing Pad)

The polishing pad that may be used in the polishing method of the present invention is not particularly limited, and it may be a pad having an unexpanded structure or a pad having an expanded structure. In pads having an unexpanded structure, a hard synthetic resin bulk material such as a plastic plate is used as the pad. There are three general types of pads having an expanded structure: those made of closed-cell foam (dry expanded), those made of open-cell foam (wet expanded), and those made of two-layer composites (laminated). Of these, pads made of two-layer composites (laminated) are especially preferred. Expansion may be uniform or non-uniform.

The polishing pad that may be used in the polishing method of the present invention may further contain the abrasive used in polishing (for example, ceria, silica, alumina, or resin). The polishing pad may be of a soft or hard nature. In a laminated polishing pad, it is preferable to have the layers made of materials of different hardnesses. Preferred materials for the polishing pad include nonwoven fabric, synthetic leather, polyamide, polyurethane, polyester and polycarbonate. The surface of the polishing pad which comes into contact with the surface to be polished may be shaped so as to form thereon, for example, grooves arranged as a grid, holes, concentric grooves, or spiral grooves.

Next, the object (substrate, wafer) to be polished by the polishing method of the present invention is described.

### (Metal Wiring Material)

The object used in the polishing method of the present invention is preferably a substrate (wafer) having interconnections made of copper or a copper alloy. Of the copper alloys, one containing silver is suitable as the metal wiring material. Beneficial effects are obtained at a silver content in the copper alloy of not more than 10% by weight and preferably not more than 1% by weight. The most beneficial effects are obtained at a silver content in the copper alloy of 0.00001 to 0.1% by weight.

### (Width of Interconnects)

In dynamic random access memory (DRAM) devices, for example, the object that may be used in the polishing method of the present invention has interconnections with a half pitch of preferably up to 0.15 μm, more preferably up to 0.10 μm, and even more preferably up to 0.08 μm.

In microprocessing unit (MPU) devices, the half pitch is preferably up to 0.12 μm, more preferably up to 0.09 μm, and even more preferably up to 0.07 μm.

The metal polishing slurry used in the present invention exhibits particularly outstanding effects on those having such interconnections.

### (Metallic Barrier Material)

In the object used in the polishing method of the present invention, a barrier layer for preventing diffusion of copper is provided between copper interconnections and an insulating film (including an interlayer dielectric film). Exemplary metallic barrier materials making up the barrier layer that may be preferably used include low-resistance metallic materials such as TiN, TiW, Ta, TaN, W and WN. Of these, Ta and TaN are particularly preferable.

### EXAMPLES

The characteristic features of the present invention are described below in further detail with reference to synthetic examples and examples. The materials, amounts of use, ratios, treatments and treatment procedures illustrated in the examples below may be modified as appropriate as long as they do not depart from the scope and spirit of the present invention. Therefore, the scope of the present invention should not be construed as being limited to the following specific examples.

### Synthetic Example 1

### Synthesis of Illustrated Compound I-7

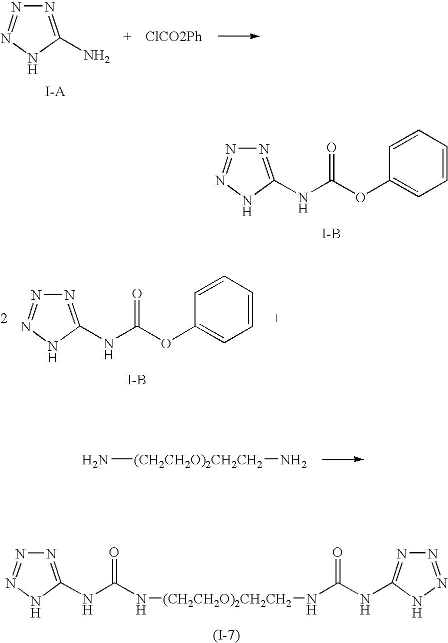

5-Aminotetrazole (I-A) (8.5 g, available from Tokyo Chemical Industry Co., Ltd.) was dissolved in N-methylpyrrolidone (200 ml), and to this solution in an ice bath, phenyl chlorocarbonate (17.1 g) was gradually added dropwise. After heating to 40° C., the reaction mixture was stirred for 2 hours. The reaction mixture was added to 2 L ice water with stirring. The precipitate was separated by suction filtration, and washed by adding water (1 L). The separated filtrate was dried to obtain I-B (18.3 g). The thus obtained I-B was dissolved in acetonitrile (100 ml), and after adding bis(2-aminoethoxy)ethane (6.5 g), the mixture was stirred at 60° C. for 3 hours. The reaction mixture was added to 1 L ice water with stirring. The precipitate was separated by suction filtration, and washed by adding water (0.5 L). The filtrate was further recrystallized with methanol to obtain I-7 (12.5 g).

### Synthetic Example 2

### Synthesis of Illustrated Compound I-12

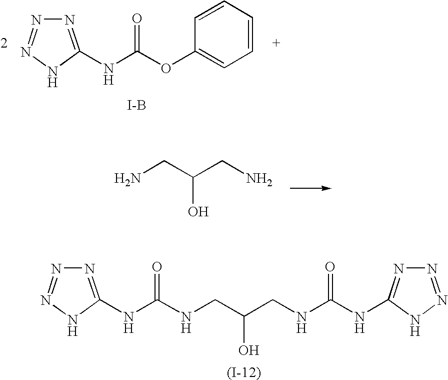

The I-B (20.5 g) obtained as above was dissolved in acetonitrile (100 ml), and after adding 1.3-diamino-2-hydroxypropane (4.5 g), the mixture was stirred at 60° C. for 3 hours. The reaction mixture was added to 1 L ice water with stirring. The precipitate was separated by suction filtration, and washed by adding water (0.5 L). The filtrate was further recrystallized with methanol to obtain I-12 (11.3 g).

### Synthetic Example 3

### Synthesis of Illustrated Compound I-4

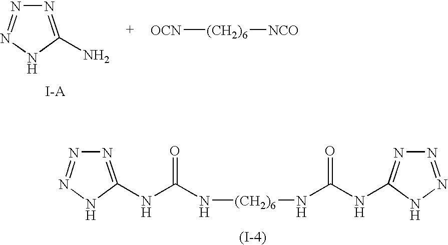

5-Aminotetrazole (I-A) (17 g, available from Tokyo Chemical Industry Co., Ltd.) was dissolved in N-methylpyrrolidone (150 ml), and to this solution was gradually added dropwise hexamethylene diisocyanate (16.8 g) at 25° C. The temperature of the reaction mixture rose to 40° C. After heating to 60° C., the mixture was stirred for 1 hour. After returning to room temperature, the reaction mixture was added to 2 L water with stirring. The precipitate was separated by suction filtration, and washed by adding water (1 L). The separated filtrate was dried to obtain I-4 (29.2 g).

### Synthetic Example 4

### Synthesis of Illustrated Compound I-11

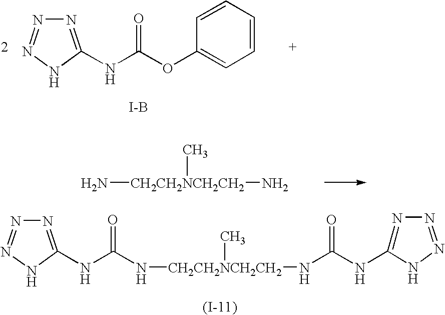

The I-B (41.0 g) obtained by the method described in Synthetic Example 1 was dissolved in acetonitrile (200 ml), and after adding bis(2-aminoethyl)methylamine (11.7 g), the mixture was stirred at 60° C. for 3 hours. The reaction mixture was added to 1 L ice water with stirring. The precipitate was separated by suction filtration, and washed by adding water (1 L). The filtrate was further recrystallized with methanol to obtain I-11 (23.5 g).

### Synthetic Example 5

### Synthesis of Illustrated Compound I-15

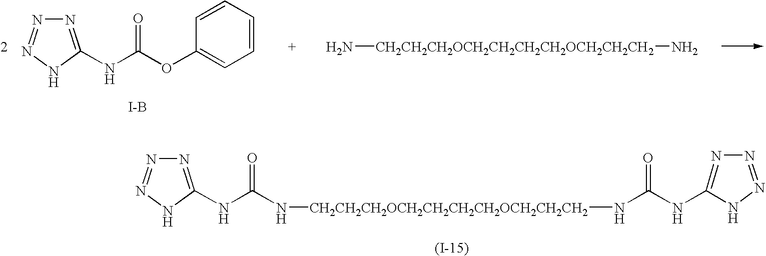

The I-B (41.0 g) obtained by the method described in Synthetic Example 1 was dissolved in acetonitrile (200 ml), and after adding bis(3-aminopropyloxy)butane (20.4 g), the mixture was stirred at 60° C. for 3 hours. The reaction mixture was added to 1 L ice water with stirring. The precipitate was separated by suction filtration, and washed by adding water (1 L). The filtrate was further recrystallized with methanol to obtain I-15 (39.0 g).

### Synthetic Example 6

### Synthesis of Illustrated Compound I-16

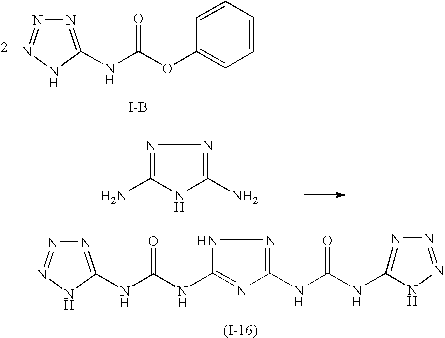

The I-B (41.0 g) obtained by the method described in Synthetic Example 1 was dissolved in N-methylpyrrolidone (150 ml), and after adding 3,5-diamino-1,2,4-triazole (9.1 g), the mixture was stirred at 80° C. for 2 hours. The reaction mixture was added to 1 L water with stirring. The precipitate was separated by suction filtration, washed by adding water (1 L) and dried to obtain I-16 (31.1 g).

### Synthetic Example 7

### Synthesis of Illustrated Compound I-18

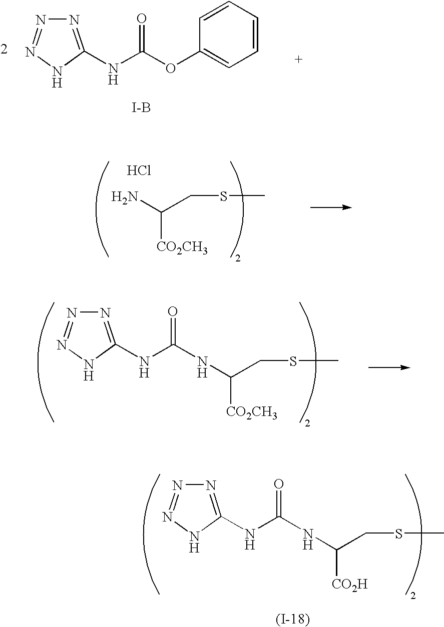

The I-B (20.5 g) obtained by the method described in Synthetic Example 1 was dissolved in acetonitrile (100 ml), and after adding cystine dimethyl ester hydrochloride (17.1 g) and triethylamine (28 ml), the mixture was stirred at 60° C. for 2 hours. The reaction mixture was added to 1 L water with stirring. The precipitate was separated by suction filtration, and washed by adding water (1 L). The resulting compound was dissolved in a mixture of methanol (100 ml) and an aqueous sodium hydroxide (100 ml) with a molar concentration of 1 and the solution was stirred at 50° C. for 3 hours. The reaction mixture was added to an aqueous hydrochloric acid (300 ml) with a molar concentration of 1. The precipitate was separated by suction filtration, washed by adding water (1 L) and dried to obtain I-18 (18.0 g).

### Synthetic Example 8

### Synthesis of Illustrated Compound I-75

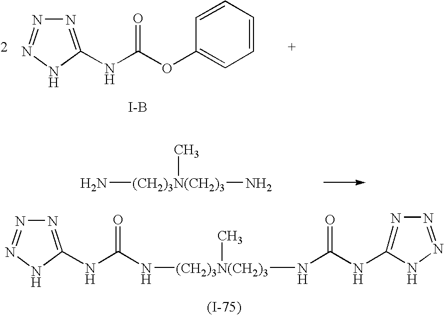

The I-B (41.0 g) obtained by the method described in Synthetic Example 1 was dissolved in acetonitrile (200 ml), and after adding bis(3-aminopropyl)methylamine (14.5 g), the mixture was stirred at 60° C. for 3 hours. The reaction mixture was added to 1 L ice water with stirring. The precipitate was separated by suction filtration, and washed by adding water (1 L). The filtrate was further recrystallized with methanol to obtain I-75 (31.8 g).

### Synthetic Example 9

### Synthesis of Illustrated Compound I-24

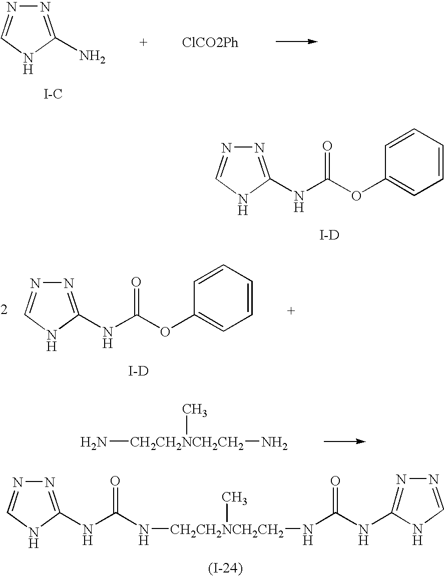

3-Amino-1,2,4-triazole (I-C) (8.4 g, available from Tokyo Chemical Industry Co., Ltd.) was dissolved in N-methylpyrrolidone (200 ml), and to this solution in an ice bath, phenyl chlorocarbonate (17.1 g) was gradually added dropwise. After heating to 40° C., the reaction mixture was stirred for 2 hours. The reaction mixture was added to 2 L ice water with stirring. The precipitate was separated by suction filtration, and washed by adding water (1 L). The separated filtrate was dried to obtain I-D (15.9 g). The thus obtained I-D was dissolved in acetonitrile (100 ml), and after adding bis(2-aminoethyl)methylamine (5.9 g), the mixture was stirred at 60° C. for 3 hours. The reaction mixture was added to 1 L ice water with stirring. The precipitate was separated by suction filtration, and washed by adding water (0.5 L). The filtrate was further recrystallized with methanol to obtain I-24 (11.5 g).

### Synthetic Example 10

### Synthesis of Illustrated Compound I-27

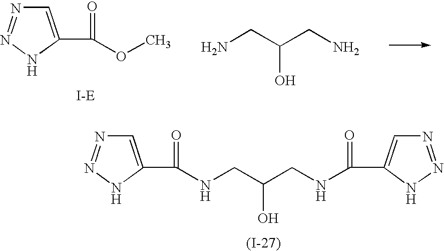

I-E (12.7 g) synthesized by the method described in a literature (Tetrahedron, vol. 61, No. 21, 2005, pp. 4983-4987) was dissolved in acetonitrile (50 ml), and after adding 1,3-diamino-2-hydroxypropane (4.5 g), the mixture was stirred at 60° C. for 3 hours. The reaction mixture was added to 1 L ice water with stirring. The precipitate was separated by suction filtration, and washed by adding water (0.5 L). The filtrate was further recrystallized with methanol to obtain I-27 (9.2 g).

### Synthetic Example 11

### Synthesis of Illustrated Compound I-30

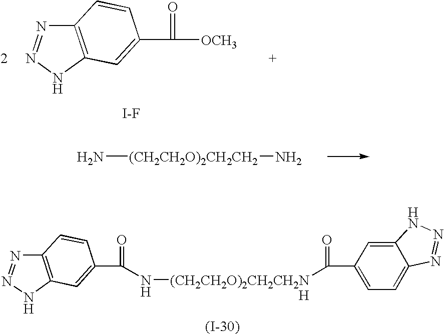

5-Aminotetrazole (I-F) (17.7 g, available from ACROS) was dissolved in N-methylpyrrolidone (200 ml). To this solution was added bis(2-aminoethoxy)ethane (7.4 g) and the mixture was stirred at 60° C. for 3 hours. The reaction mixture was added to 1 L ice water with stirring. The precipitate was separated by suction filtration, and washed by adding water (0.5 L). The filtrate was further recrystallized with methanol to obtain I-30 (16.9 g).

### Synthetic Example 12

### Synthesis of Illustrated Compound I-36

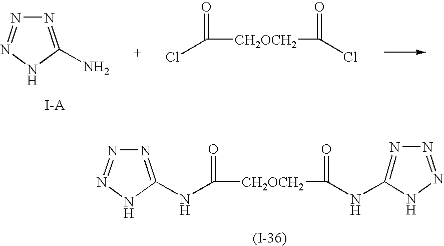

5-Aminotetrazole (I-A) (17 g, available from Tokyo Chemical Industry Co., Ltd.) was dissolved in acetonitrile (100 ml), and to this solution in an ice bath was gradually added dropwise diglycolyl chloride (17.1 g, available from Aldrich). After heating to 60° C., the reaction mixture was stirred for 1 hour. After returning to room temperature, the reaction mixture was added to 2 L water with stirring. The precipitate was separated by suction filtration, and washed by adding water (1 L). The separated filtrate was dried to obtain I-36 (21.2 g).

### Synthetic Example 13

### Synthesis of Illustrated Compound I-44

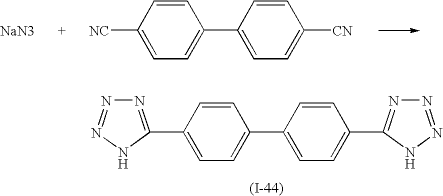

4,4′-Dicyanobiphenyl (20.4 g) and sodium azide (6.5 g) were heated in a toluene solvent and stirred for 3 hours. After returning to room temperature, the reaction mixture was added to 2 L water with stirring. The precipitate was separated by suction filtration, and washed by adding water (1 L). The separated filtrate was dried to obtain I-44 (18.0 g).

### Synthetic Example 14

### Synthesis of Illustrated Compound I-47

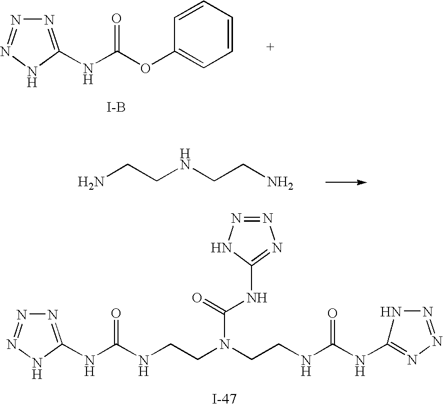

The I-B (15.0 g) obtained by the method described in Synthetic Example 1 was dissolved in NMP (70 ml), and after adding diethylenetriamine (0.5 g available from Tokyo Chemical Industry Co., Ltd.), the mixture was stirred at 100° C. for 3 hours. The reaction mixture was added to 0.6 L water with stirring. The precipitate was separated by suction filtration and washed by adding water (0.4 L) and acetonitrile (0.4 L) to obtain I-47 (9.0 g) as a colorless solid.

### Synthetic Example 15

### Synthesis of Illustrated Compound I-52

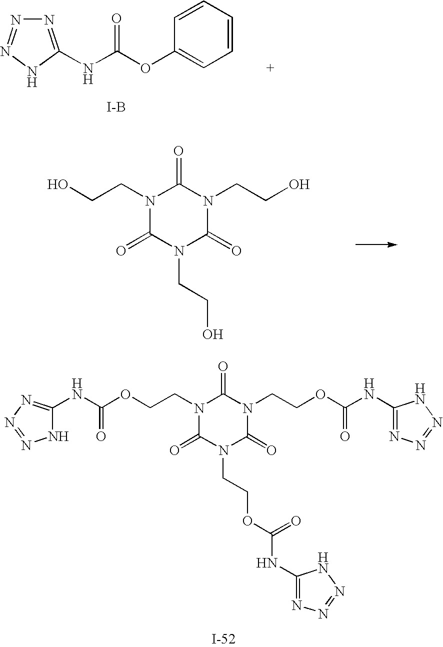

The I-B (15.0 g) obtained by the method described in Synthetic Example 1 was dissolved in NMP (90 ml), and after adding tris(2-hydroxyethyl)isocyanurate (6.36 g available from Tokyo Chemical Industry Co., Ltd.), the mixture was stirred at 100° C. for 2 hours. The reaction mixture was added to 0.5 L water with stirring. The precipitate was separated by suction filtration and washed by adding water (0.3 L) and acetonitrile (0.3 L) to obtain I-52 (10.8 g) as a colorless solid.

### Synthetic Example 16

### Synthesis of Illustrated Compound I-53

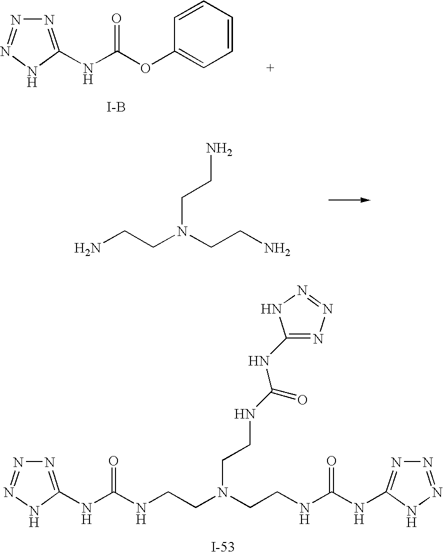

The I-B (12.3 g) obtained by the method described in Synthetic Example 1 was dissolved in NMP (70 ml), and after adding tris(2-aminoethyl)amine (3.0 mL available from Tokyo Chemical Industry Co., Ltd.), the mixture was stirred at 100° C. for 2 hours. The reaction mixture was added to 0.5 L water with stirring. The precipitate was separated by suction filtration and washed by adding water (0.3 L) and acetonitrile (0.3 L) to obtain I-53 (14.3 g) as a colorless solid.

### Synthetic Example 17

### Synthesis of Illustrated Compound I-49

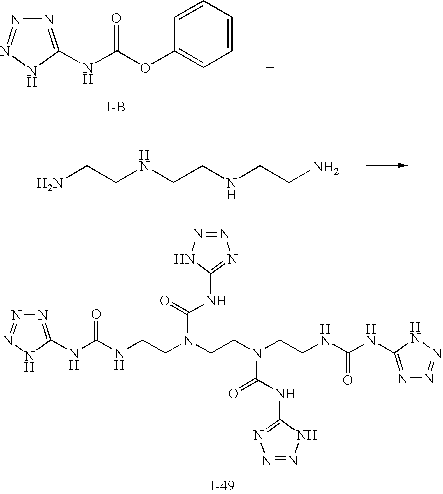

The I-B (42.8 g) obtained by the method described in Synthetic Example 1 was dissolved in NMP (200 ml), and after adding triethylenetetramine (7.6 g available from Tokyo Chemical Industry Co., Ltd.), the mixture was stirred at 100° C. for 1 hour. The reaction mixture was added to 0.4 L water with stirring. The precipitate was separated by suction filtration and washed by adding water (0.4 L) and acetonitrile (0.4 L) to obtain I-49 (23.0 g) as a colorless solid.

### Synthetic Example 18

### Synthesis of Illustrated Compound I-48

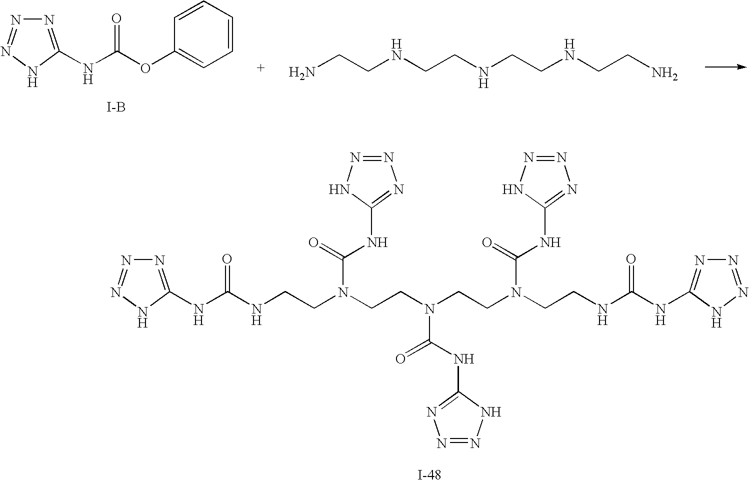

The I-B (56.0 g) obtained by the method described in Synthetic Example 1 was dissolved in NMP (250 ml), and after adding tetraethylenepentamine (10.35 g available from Tokyo Chemical Industry Co., Ltd.), the mixture was stirred at 100° C. for 1 hour. The reaction mixture was added to 1.5 L water with stirring. The precipitate was separated by suction filtration and washed by adding water (1 L) and acetonitrile (1 L) to obtain I-48 (37.0 g) as a colorless solid.

### Examples 1 to 55 and Comparative Examples 1 to 6

Polishing slurry Nos. 101 to 155 and 201 to 206 shown in Tables 1 to 7 below were prepared for evaluation by polishing test.

### (Preparation of Metal Polishing Slurry)

Each metal polishing slurry was prepared by mixing the following ingredients.

| | |
|---|---|
| the compound of the invention: the compound | 1.5 mmol/L |
| shown in Tables 1 to 7 | |
| organic acid: the compound shown in | 0.26 mol/L |
| Tables 1 to 7 | |
| abrasive: the abrasive shown in Tables 1 to 7 | 3.2 g/L |
| surfactant: the compound shown in Tables 1 to 7 | 0.01 g/L |
| oxidizer: hydrogen peroxide | 12.5 g/L |

Pure water was added to a total volume of 1000 mL, and the pH was adjusted with ammonia solution to pH 7.0.

Commercially available colloidal silica was used in all Examples and Comparative Examples. The colloidal silica used had a primary particle size (simply referred to as “particle size” in the tables) of 20 to 70 nm.

### (Polishing Test)

Polishing was conducted under the following conditions to evaluate the polishing rate and the resistance to dishing.
Polisher: FREX 300 (manufactured by Ebara Corporation)
Polishing object (wafer):
(1) The wafer for evaluating the polishing rate: a blanket wafer having a diameter of 300 mm prepared by forming a copper film having a thickness of 1.5 μm on a silicon substrate;
(2) The wafer for evaluating the resistance to dishing: copper wiring wafer having a diameter of 300 mm (patterned wafer: mask pattern 754 CMP manufactured by ATDF)
Polishing pad: IC14⁰0-K Groove (manufactured by Rodel)
Polishing condition:
Polishing pressure (contact pressure between the surface to be polished and the polishing pad): 14000 Pa
Feed rate of the polishing slurry: 200 ml/min
Rotation speed of polishing platen: 104 rpm
Rotation speed of polishing head: 85 rpm

### (Evaluation Method)

Calculation of the polishing rate: The blanket wafer in (1) was polished for 60 seconds, and the thickness of the metal film before and after polishing was determined from the electrical resistance at evenly distributed 49 locations on the surface of the wafer. The average of the thickness changes divided by the polishing time was defined as the polishing rate.

Evaluation of dishing resistance: The patterned wafer in (2) was polished for a period of time including the time required for completely polishing copper of non-wiring portions and the time corresponding to 25% of the foregoing polishing time. The step height of line-and-space portions (line 10 μm, space 10 μm) was measured with a contact profilometer Dektak V3201 manufactured by Veeco Instruments.

Evaluation of scratch count: The polished copper film was evaluated by counting the number of defects in the entire polished area using a wafer inspection apparatus (ComPLUS manufactured by Applied Materials). Next, 200 defects were randomly chosen from the defects detected by the wafer inspection apparatus, and scratch defects were counted in these 200 defects. The number of scratches (scratch count) on the entire wafer surface was estimated by the following equation.

Scratch count (count/surface)=Number of all defects detected by the wafer inspection apparatus (count/surface)×{(number of scratch defects in the 200 chosen defects)/200}

The evaluation results are shown in Tables 1 to 7.

TABLE 1

| | Polishing slurry | | | | | Results of evaluation | | |
|---|---|---|---|---|---|---|---|---|
| | | | | Abrasive | | Polishing | | |
| | | Passivation film- | | (particle size, | | rate | Dishing | Scratch count |
| | Type | forming agent | Organic acid | association degree) | Surfactant | (Å/min) | (Å) | (count/surface) |
| Ex. 1 | 101 | Inventive compound | Glycine | Colloidal silica | Newcol 220L | 7300 | 550 | 43 |
| | | I-4 | | (40 nm, 2) | | | | |
| Ex. 2 | 102 | Inventive compound | Glycine | Colloidal silica | Newcol 220L | 7350 | 390 | 17 |
| | | I-7 | | (40 nm2) | | | | |
| Ex. 3 | 103 | Inventive compound | α-Alanine | Colloidal silica | NEOPELEX No. 6 | 7250 | 370 | 19 |
| | | I-7 | | (40 nm2) | | | | |
| Ex. 4 | 104 | Inventive compound | N-Methylglycine | Colloidal silica | ELEMINOL MON | 7200 | 360 | 16 |
| | | I-7 | | (20 nm1) | | | | |
| Ex. 5 | 105 | Inventive compound | Glycine | Colloidal silica | DOWFAX 2A-1 | 7450 | 430 | 20 |
| | | I-11 | | (30 nm1) | | | | |
| Ex. 6 | 106 | Inventive compound | Glycine | Colloidal silica | NEOPELEX No. 6 | 7500 | 490 | 23 |
| | | I-11 | | (30 nm1) | | | | |
| Ex. 7 | 107 | Inventive compound | Glycine | Colloidal silica | — | 7650 | 620 | 45 |
| | | I-11 | | (30 nm1) | | | | |
| Ex. 8 | 108 | Inventive compound | Oxalic acid | Colloidal silica | NEOPELEX No. 6 | 6950 | 630 | 53 |
| | | I-11 | | (30 nm1) | | | | |
| Ex. 9 | 109 | Inventive compound | α-Alanine | Colloidal silica | Pionine A-43-N | 7250 | 350 | 13 |
| | | I-12 | | (20 nm2) | | | | |

TABLE 2

| | Polishing slurry | | | | | Results of evaluation | | |
|---|---|---|---|---|---|---|---|---|
| | | | | Abrasive | | Polishing | | |
| | | Passivation film- | | (particle size, | | rate | Dishing | Scratch count |
| | Type | forming agent | Organic acid | association degree) | Surfactant | (Å/min) | (Å) | (count/surface) |
| Ex. 10 | 110 | Inventive | N-Methyl- | Colloidal silica | Pionine A-43S | 7100 | 310 | 15 |
| | | compound I-12 | glycine | (30 nm, 2) | | | | |
| Ex. 11 | 111 | Inventive | Hydroxy- | Colloidal silica | Pionine A-41 | 7150 | 350 | 15 |
| | | compound I-12 | ethylglycine | (40 nm, 1) | | | | |
| Ex. 12 | 112 | Inventive | Dihydroxy- | Colloidal silica | PELEX SS-L | 7200 | 340 | 13 |
| | | compound I-12 | ethylglycine | (40 nm, 2) | | | | |
| Ex. 13 | 113 | Inventive | α-Alanine | Colloidal silica | PELEX SS-L | 7200 | 440 | 24 |
| | | compound I-15 | | (40 nm, 2) | | | | |
| Ex. 14 | 114 | Inventive | Dihydroxy- | Colloidal silica | PELEX SS-L | 7150 | 430 | 21 |
| | | compound I-15 | ethylglycine | (40 nm, 2) | | | | |
| Ex. 15 | 115 | Inventive | Glycine | Colloidal silica | PELEX SS-L | 7550 | 590 | 36 |
| | | compound I-16 | | (20 nm, 1) | | | | |
| Ex. 16 | 116 | Inventive | Glycine | Colloidal silica | PELEX SS-L | 7800 | 660 | 38 |
| | | compound I-16 | | (70 nm, 3) | | | | |
| Ex. 17 | 117 | Inventive | Tricine | Colloidal silica | PELEX SS-L | 7100 | 530 | 32 |
| | | compound I-18 | | (40 nm, 2) | | | | |
| Ex. 18 | 118 | Inventive | α-Alanine | Colloidal silica | PELEX SS-L | 7250 | 550 | 35 |
| | | compound I-18 | | (40 nm, 2) | | | | |

TABLE 3

| | Polishing slurry | | | | | Results of evaluation | | |
|---|---|---|---|---|---|---|---|---|
| | | | | Abrasive | | Polishing | | |
| | | Passivation film- | | (particle size, association | | rate | Dishing | Scratch count |
| | Type | forming agent | Organic acid | degree) | Surfactant | (Å/min) | (Å) | (count/surface) |
| Ex. 19 | 119 | Inventive | Glycine | Colloidal silica | PELEX SS-L | 7800 | 420 | 19 |
| | | compound I-19 | | (40 nm, 2) | | | | |
| Ex. 20 | 120 | Inventive | Glycine | Colloidal silica | PELEX SS-L | 7450 | 520 | 29 |
| | | compound I-24 | | (40 nm, 2) | | | | |
| Ex. 21 | 121 | Inventive | β-Alanine | Colloidal silica | DIAPON K | 6950 | 470 | 30 |
| | | compound I-24 | | (40 nm, 2) | | | | |
| Ex. 22 | 122 | Inventive | Glycine | Colloidal silica | PELEX SS-H | 7500 | 510 | 26 |
| | | compound I-27 | | (40 nm, 2) | | | | |
| Ex. 23 | 123 | Inventive | Iminodiacetic | Colloidal silica | IONET D-2 | 7700 | 620 | 28 |
| | | compound I-27 | acid | (40 nm, 2) | | | | |
| Ex. 24 | 124 | Inventive | Alanine- | Colloidal silica | DEMOL SS-L | 7350 | 600 | 42 |
| | | compound I-30 | diacetic acid | (30 nm, 1) | | | | |
| Ex. 25 | 125 | Inventive | Methylglycine- | Colloidal silica | Solvalite C-45 | 7550 | 590 | 39 |
| | | compound I-36 | diacetic acid | (40 nm, 2) | | | | |
| Ex. 26 | 126 | Inventive | Glycine | Colloidal silica | Newcol 220L | 5200 | 650 | 75 |
| | | compound I-44 | | (40 nm, 2) | | | | |

TABLE 4

| | Polishing slurry | | | | | Results of evaluation | | |
|---|---|---|---|---|---|---|---|---|
| | | | | Abrasive | | Polishing | | |
| | | Passivation film- | | (particle size, | | rate | Dishing | Scratch count |
| | Type | forming agent | Organic acid | association degree) | Surfactant | (Å/min) | (Å) | (count/surface) |
| Ex. 27 | 127 | Inventive compound | Glycine | Colloidal silica | PELEX SS-L | 8150 | 320 | 18 |
| | | I-47 | | (40 nm, 2) | | | | |
| Ex. 28 | 128 | Inventive compound | β-Alanine | Colloidal silica | PELEX SS-L | 7950 | 330 | 24 |
| | | I-47 | | (40 nm, 2) | | | | |
| Ex. 29 | 129 | Inventive compound | Glycine | Colloidal silica | PELEX SS-L | 8650 | 305 | 22 |
| | | I-48 | | (40 nm, 2) | | | | |
| Ex. 30 | 130 | Inventive compound | N-Methyl- | Colloidal silica | PELEX SS-L | 8300 | 330 | 28 |
| | | I-49 | glycine | (40 nm, 2) | | | | |
| Ex. 31 | 131 | Inventive compound | Glycine | Colloidal silica | Pionine A-41 | 7900 | 325 | 32 |
| | | I-50 | | (40 nm, 1) | | | | |
| Ex. 32 | 132 | Inventive compound | β-Alanine | Colloidal silica | Newcol 220L | 8200 | 310 | 36 |
| | | I-51 | | (40 mn, 2) | | | | |
| Ex. 33 | 133 | Inventive compound | β-Alanine | Colloidal silica | IONET D-2 | 7900 | 335 | 21 |
| | | I-52 | | (40 nm, 2) | | | | |
| Ex. 34 | 134 | Inventive compound | N-Methyl- | Colloidal silica | Newcol 220L | 8150 | 310 | 21 |
| | | I-53 | glycine | (40 nm, 2) | | | | |
| Ex. 35 | 135 | Inventive compound | Glycine | Colloidal silica | NEOPELEX No. 6 | 8050 | 320 | 26 |
| | | I-54 | | (40 nm, 2) | | | | |

TABLE 5

| | Polishing slurry | | | | | Results of evaluation | | |
|---|---|---|---|---|---|---|---|---|
| | | | | Abrasive | | Polishing | | |
| | | Passivation film- | | (particle size, | | rate | Dishing | Scratch count |
| | Type | forming agent | Organic acid | association degree) | Surfactant | (Å/min) | (Å) | (count/surface) |
| Ex. 36 | 136 | Inventive compound | Glycine | Colloidal silica | NEOPELEX No. 6 | 8150 | 350 | 32 |
| | | I-55 | | (40 nm, 2) | | | | |
| Ex. 37 | 137 | Inventive compound | Glycine | Colloidal silica | NEOPELEX No. 6 | 6700 | 350 | 37 |
| | | I-56 | | (40 nm, 2) | | | | |
| Ex. 38 | 138 | Inventive compound | Glycine | Colloidal silica | Newcol 220L | 8200 | 320 | 22 |
| | | I-57 | | (40 nm, 2) | | | | |
| Ex. 39 | 139 | Inventive compound | N-Methyl- | Colloidal silica | IONET D-2 | 8000 | 330 | 28 |
| | | I-58 | glycine | (40 nm, 2) | | | | |
| Ex. 40 | 140 | Inventive compound | N-Methyl- | Colloidal silica | IONET D-2 | 8100 | 330 | 23 |
| | | I-59 | glycine | (40 nm, 2) | | | | |
| Ex. 41 | 141 | Inventive compound | β-Alanine | Colloidal silica | PELEX SS-L | 8050 | 340 | 24 |
| | | I-60 | | (30 nm, 1) | | | | |
| Ex. 42 | 142 | Inventive compound | Glycine | Colloidal silica | PELEX SS-L | 8400 | 315 | 27 |
| | | I-61 | | (40 nm, 2) | | | | |
| Ex. 43 | 143 | Inventive compound | Glycine | Colloidal silica | Newcol 220L | 8100 | 320 | 32 |
| | | I-62 | | (40 nm, 2) | | | | |
| Ex. 44 | 144 | Inventive compound | N-Methyl- | Colloidal silica | NEOPELEX No. 6 | 8200 | 325 | 19 |
| | | I-63 | glycine | (40 nm, 2) | | | | |
| Ex. 45 | 145 | Inventive compound | Glycine | Colloidal silica | PELEX SS-L | 8150 | 340 | 25 |
| | | I-64 | | (40 nm, 2) | | | | |

TABLE 6

| | Polishing slurry | | | | | Results of evaluation | | |
|---|---|---|---|---|---|---|---|---|
| | | | | Abrasive | | Polishing | | |
| | | Passivation film- | | (particle size, | | rate | Dishing | Scratch count |
| | Type | forming agent | Organic acid | association degree) | Surfactant | (Å/min) | (Å) | (count/surface) |
| Ex. 46 | 146 | Inventive compound | β-Alanine | Colloidal silica | PELEX SS-L | 8300 | 335 | 18 |
| | | I-65 | | (40 nm, 2) | | | | |
| Ex. 47 | 147 | Inventive compound | β-Alanine | Colloidal silica | Pionine A-43-N | 8150 | 325 | 26 |
| | | I-66 | | (40 nm, 2) | | | | |
| Ex. 48 | 148 | Inventive compound | Glycine | Colloidal silica | Pionine A-43-N | 8300 | 320 | 25 |
| | | I-67 | | (40 nm, 2) | | | | |
| Ex. 49 | 149 | Inventive compound | Glycine | Colloidal silica | NEOPELEX No. 6 | 8350 | 340 | 31 |
| | | I-68 | | (40 nm, 2) | | | | |
| Ex. 50 | 150 | Inventive compound | β-Alanine | Colloidal silica | IONET D-2 | 8150 | 325 | 33 |
| | | I-69 | (40 nm, 2) | | | | | |
| Ex. 51 | 151 | Inventive compound | Glycine | Colloidal silica | PELEX SS-L | 8300 | 330 | 23 |
| | | I-70 | | (30 nm, 1) | | | | |
| Ex. 52 | 152 | Inventive compound | Glycine | Colloidal silica | PELEX SS-L | 8350 | 335 | 26 |
| | | I-71 | | (40 nm, 2) | | | | |
| Ex. 53 | 153 | Inventive compound | Glycine | Colloidal silica | PELEX SS-L | 8050 | 320 | 33 |
| | | I-72 | | (40 nm, 2) | | | | |
| Ex. 54 | 154 | Inventive compound | N-Methyl- | Colloidal silica | Pionine A-41 | 8400 | 320 | 25 |
| | | I-73 | glycine | (40 nm, 2) | | | | |
| Ex. 55 | 155 | Inventive compound | N-Methyl- | Colloidal silica | PELEX SS-L | 8350 | 325 | 29 |
| | | I-74 | glycine | (40 nm, 2) | | | | |

TABLE 7

| | Polishing slurry | | | | | Results of evaluation | | |
|---|---|---|---|---|---|---|---|---|
| | | | | Abrasive | | Polishing | | |
| | | Passivation film- | | (particle size, | | rate | Dishing | Scratch count |
| | Type | forming agent | Organic acid | association degree) | Surfactant | (Å/min) | (Å) | (count/surface) |
| Comp. | 201 | Comparative | Glycine | Colloidal silica | PELEX SS-L | 2500 | 1250 | 254 |
| Ex. 1 | | compound 1 | | (40 nm, 2) | | | | |
| Comp. | 202 | Comparative | α-Alanine | Colloidal silica | NEOPELEX No. 6 | 1750 | 1450 | 266 |
| Ex. 2 | | compound 1 | | (40 nm, 2) | | | | |
| Comp. | 203 | Comparative | Hydroxy- | Colloidal silica | Pionine A-41 | 1550 | 1800 | 221 |
| Ex. 3 | | compound 1 | ethylglycine | (40 nm, 1) | | | | |
| Comp. | 204 | Comparative | Glycine | Colloidal silica | PELEX SS-L | 7650 | 780 | 162 |
| Ex. 4 | | compound 2 | | (40 nm, 2) | | | | |
| Comp. | 205 | Comparative | α-Alanine | Colloidal silica | Pionine A-43-N | 7250 | 750 | 173 |
| Ex. 5 | | compound 2 | | (40 nm, 2) | | | | |
| Comp. | 206 | Comparative | Dihydroxy- | Colloidal silica | PELEX SS-L | 7100 | 700 | 119 |
| Ex. 6 | | compound 2 | ethylglycine | (40 nm, 2) | | | | |

Newcol 220L: sodium dodecylbenzene sulfonate available from Nippon Nyukazai Co., Ltd.
NEOPELEX No. 6: dodecylbenzene sulfonate available from Kao Corporation
ELEMINOL MON: sodium dodecylalkyl diphenyl ether disulfonate available from Sanyo Chemical Industries, Ltd.
DOWFAX 2A-1: sodium dodecylalkyl diphenyl ether disulfonate available from DOW
PELEX SS-L: sodium alkyl diphenyl ether disulfonate available from Kao Corporation
Pionine A-43-N: ammonium dodecylalkyl diphenyl ether disulfonate available from Takemoto Oil and Fat Co., Ltd.
Pionine A-43S: sodium dodecyl diphenyl ether disulfonate available from Takemoto Oil and Fat Co., Ltd.
Pionine A-41: dodecylbenzene sulfonate available from Takemoto Oil and Fat Co., Ltd.
DIAPON K: sodium acyl (coconut) methyltaurate available from NOF Corporation
PELEX SS-H: sodium alkyl diphenyl ether disulfonate available from Kao Corporation
IONET D2: sodium salt of a naphthalenesulfonic acid/formalin condensate available from Sanyo Chemical Industries,
Ltd.
DEMOL SS-L: sodium salt of a specific aromatic sulfonic acid/formalin condensate available from Kao Corporation
Solvalite C45: sodium salt of a naphthalenesulfonic acid/formalin condensate
Comparative compound 1

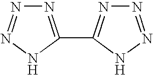

Comparative compound 2

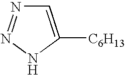

As is clear from the results shown in Tables 1 to 7, a high polishing rate and reduced dishing could be simultaneously achieved by the chemical mechanical polishing method using the metal polishing slurry of the present invention. The scratch count was also low, and the superiority of the compound of the present invention was thereby confirmed.

It was revealed that the advantage of the present invention was particularly significant when the surfactant used was dodecyl diphenyl ether disulfonate, when the organic acid was an amino acid, and when the abrasive had a particle size of 20 to 40 nm.

### Examples 56 to 73 and Comparative Examples 7 to 12

Polishing slurry Nos. 156 to 173 shown in Tables 8 to 10 were prepared for evaluation by polishing test. The following numbers of the compounds of the present invention correspond to the numbers of the compounds represented by the general formula (1) or (3) as described above.

### (Preparation of Metal Polishing Slurry)

Each metal polishing slurry was prepared by mixing the following ingredients.

| | |
|---|---|
| the compound of the invention: the compound | 1.5 mmol/L |
| shown in Tables 8 to 10 | |
| organic acid: the compound shown in Tables | 0.26 mol/L |
| 8 to 10 | |
| abrasive: the abrasive shown in Tables 8 to 10 | 3.2 g/L |
| surfactant: the compound shown in Tables | 0.01 g/L |
| 8 to 10 | |
| oxidizer: hydrogen peroxide | 12.5 g/L |

Pure water was added to a total volume of 1000 mL, and the pH was adjusted with ammonia solution to pH 7.0.

Commercially available colloidal silica was used in all Examples and Comparative Examples. The colloidal silica had a primary particle size (simply referred to as “particle size” in the tables) of 20 to 70 nm.

### (Polishing Test 2)

Polishing was conducted under the following conditions to evaluate the polishing rate and the resistance to dishing.
Polisher: Reflexion (manufactured by Applied Materials)
Polishing object (wafer):
(1) The wafer for evaluating the polishing rate: a blanket wafer having a diameter of 300 mm prepared by forming a copper film having a thickness of 1.5 μm on a silicon substrate;
(2) The wafer for evaluating the resistance to dishing: copper wiring wafer having a diameter of 300 mm (patterned wafer: mask pattern 754 CMP manufactured by ATDF)
Polishing pad: IC1010 (manufactured by Rodel)
Polishing condition:
Polishing pressure (contact pressure between the surface to be polished and the polishing pad): 7000 Pa
Feed rate of the polishing slurry: 200 ml/min
Rotation speed of polishing platen: 110 rpm
Rotation speed of polishing head: 100 rpm
Polishing method: In this polishing test, the patterned wafer in (2) was polished by a polishing method including first and second polishing steps:

first polishing step: the step of polishing a conductor film made of copper or a copper alloy to the residual thickness of 2000 Å,

second polishing step: the step of polishing for a period of time including the time required for completely removing the copper of the non-wiring portions by polishing and the time corresponding to 25% of the foregoing polishing time.

### (Evaluation Method)

Calculation of the polishing rate: The blanket wafer in (1) was polished for 60 seconds, and the thickness of the metal film before and after polishing was determined from the electrical resistance at evenly distributed 49 locations on the surface of the wafer. The average of the thickness changes divided by the polishing time was defined as the polishing rate.

Evaluation of step height: The patterned wafer in (2) was polished until the copper film had a residual thickness of 2000 Å. The step height of a copper film deposited in line-and-space portions (line 10 μm, space 10 μm) was measured with a contact profilometer Dektak V3201 manufactured by Veeco Instruments.

Evaluation of dishing resistance: The patterned wafer in (2) was polished for a period of time including the time required for completely polishing copper of non-wiring portions and the time corresponding to 25% of the foregoing polishing time. The step height of line-and-space portions (line 10 μm, space 10 μm) was measured with a contact profilometer Dektak V3201 manufactured by Veeco Instruments.

Evaluation of scratch count: The polished copper film was evaluated by counting the number of defects in the entire polished area using a wafer inspection apparatus (ComPLUS manufactured by Applied Materials). Next, 200 defects were randomly chosen from the defects detected by the wafer inspection apparatus, and scratch defects were counted in these 200 defects. The number of scratches (scratch count) on the entire wafer surface was estimated by the following equation.

Scratch count (count/surface)=Number of all defects detected by the wafer inspection apparatus (count/surface)×{(number of scratch defects in the 200 chosen defects)/200}

The evaluation results are shown in Tables 8 to 10.

TABLE 8

| | Polishing slurry | | | | | Results of evaluation | | | | |
|---|---|---|---|---|---|---|---|---|---|---|
| | | | | Abrasive | | First polishing step | | Second polishing step | | |
| | | General | | (particle size, | | Polishing | | Polishing | | Scratch count |
| | | formula | Organic | average association | | rate | Step height | rate | Dishing | (count/ |
| | Type | (1) | acid | degree) | Surfactant | (Å/min) | (Å) | (Å/min) | (Å) | surface) |
| Ex. 56 | 156 | Inventive | Glycine | Colloidal silica | Newcol 220L | 7300 | 250 | 5600 | 350 | 20 |
| | | compound I-4 | | (40 nm, 2) | | | | | | |
| Ex. 57 | 157 | Inventive | Glycine | Colloidal silica | Newcol 220L | 7350 | 180 | 5500 | 370 | 18 |
| | | compound I-7 | | (40 nm, 2) | | | | | | |
| Ex. 58 | 158 | Inventive | Glycine | Colloidal silica | DOWFAX 2A-1 | 7450 | 320 | 5450 | 320 | 18 |
| | | compound I-11 | | (30 nm, 1) | | | | | | |
| Ex. 59 | 159 | Inventive | α- | Colloidal silica | Pionine A-43S | 7250 | 220 | 6250 | 500 | 32 |
| | | compound I-12 | Alanine | (20 nm, 2) | | | | | | |
| Ex. 60 | 160 | Inventive | Glycine | Colloidal silica | Newcol 220L | 7850 | 320 | 6700 | 320 | 15 |
| | | compound I-23 | | (40 nm, 2) | | | | | | |
| Ex. 61 | 161 | Inventive | N- | Colloidal silica | PELEX SS-L | 7350 | 420 | 6250 | 320 | 18 |
| | | compound I-23 | Methyl- | (40 nm, 2) | | | | | | |
| | | | glycine | | | | | | | |
| Ex. 62 | 162 | Inventive | Oxalic | Colloidal silica | NEOPELEX No. 6 | 5200 | 270 | 4950 | 500 | 32 |
| | | compound I-34 | acid | (40 nm, 2) | | | | | | |
| Ex. 63 | 163 | Inventive | N- | Colloidal silica | ELEMINOL MON | 6800 | 240 | 5000 | 450 | 20 |
| | | compound I-34 | Methyl- | (20 nm, 1) | | | | | | |
| | | | glycine | | | | | | | |
| Ex. 64 | 164 | Inventive | Tricine | Colloidal silica | ELEMINOL MON | 6750 | 170 | 5650 | 270 | 18 |
| | | compound I-45 | | (20 nm, 2) | | | | | | |
| Ex. 65 | 165 | Inventive | Oxalic | Colloidal silica | ELEMINOL MON | 6450 | 305 | 5800 | 530 | 35 |
| | | compound I-45 | acid | (70 nm, 2) | | | | | | |

TABLE 9

| | Polishing slurry | | | | | | | | | |
|---|---|---|---|---|---|---|---|---|---|---|
| | | | | Abrasive | | Results of evaluation | | | | |
| | | | | (particle size, | | First polishing step | | Second polishing step | | |
| | | | | average | | Polishing | | Polishing | | Scratch count |
| | | General formula | | association | | rate | Step | rate | Dishing | (count/ |
| | Type | (1) | Organic acid | degree) | Surfactant | (Å/min) | height (Å) | (Å/min) | (Å) | surface) |
| Ex. 66 | 166 | Inventive | Glycine | Colloidal silica | DOWFAX 2A-1 | 7200 | 310 | 6600 | 320 | 15 |
| | | compound I-46 | | (30 nm, 1) | | | | | | |
| Ex. 67 | 167 | Inventive | α-Alanine | Colloidal silica | Pionine A-43S | 7150 | 270 | 6250 | 420 | 27 |
| | | compound I-46 | | (30 nm, 1) | | | | | | |
| Ex. 68 | 168 | Inventive | Hydroxy- | Colloidal silica | Newcol 220L | 6350 | 350 | 5770 | 500 | 25 |
| | | compound I-54 | ethylglycine | (30 nm, 1) | | | | | | |
| Ex. 69 | 169 | Inventive | Tricine | Colloidal silica | Pionine A-43S | 7350 | 420 | 7000 | 490 | 18 |
| | | compound I-54 | | (30 nm, 1) | | | | | | |
| Ex. 70 | 170 | Inventive | Dihydroxy- | Colloidal silica | — | 6300 | 550 | 4950 | 680 | 55 |
| | | compound I-61 | ethylglycine | (30 nm, 1) | | | | | | |
| Ex. 71 | 171 | Inventive | N-Methyl- | Colloidal silica | NEOPELEX No. 6 | 7000 | 10 | 5200 | 350 | 20 |
| | | compound I-61 | glycine | (30 nm, 1) | | | | | | |
| Ex. 72 | 172 | Inventive | α-Alanine | Colloidal silica | Pionine A-43-N | 6550 | 140 | 5250 | 270 | 18 |
| | | compound I-68 | | (20 nm, 2) | | | | | | |
| Ex. 73 | 173 | Inventive | Hydroxy- | Colloidal silica | Pionine A-43S | 6250 | 275 | 5400 | 330 | 32 |
| | | compound I-68 | ethylglycine | (30 nm, 2) | | | | | | |

TABLE 10

| | | | | | | Results of evaluation | | | | |
|---|---|---|---|---|---|---|---|---|---|---|
| | Polishing slurry | | | | | First polishing step | | Second polishing step | | |
| | | Passivation | | Abrasive | | Polishing | | Polishing | | Scratch count |
| | | film-forming | | (particle size, | | rate | Step height | rate | Dishing | (count/ |
| | Type | agent | Organic acid | association degree) | Surfactant | (Å/min) | (Å) | (Å/min) | (Å) | surface) |
| Comp. | 201 | Comparative | Glycine | Colloidal silica | Pionine A-43-N | 5900 | 430 | 4800 | 630 | 142 |
| Ex. 7 | | compound 1 | | (20 nm, 2) | | | | | | |
| Comp. | 202 | Comparative | α-Alanine | Colloidal silica | Pionine A-43-N | 5550 | 330 | 5200 | 620 | 120 |
| Ex. 8 | | compound 1 | | (20 nm, 2) | | | | | | |
| Comp. | 203 | Comparative | Hydroxy- | Colloidal silica | Newcol 220L | 5450 | 500 | 4800 | 650 | 85 |
| Ex. 9 | | compound 1 | ethylglycine | (40 nm, 2) | | | | | | |
| Comp. | 204 | Comparative | Glycine | Colloidal silica | Pionine A-43S | 4550 | 450 | 3850 | 730 | 110 |
| Ex. 10 | | compound 2 | | (40 nm, 2) | | | | | | |
| Comp. | 205 | Comparative | α-Alanine | Colloidal silica | Pionine A-43S | 4350 | 520 | 3950 | 715 | 130 |
| Ex. 11 | | compound 2 | | (20 nm, 1) | | | | | | |
| Comp. | 206 | Comparative | Dihydroxy- | Colloidal silica | Newcol 220L | 4100 | 460 | 3200 | 680 | 90 |
| Ex. 12 | | compound 2 | ethylglycine | (40 nm, 2) | | | | | | |

Newcol 220L: sodium dodecylbenzene sulfonate available from Nippon Nyukazai Co., Ltd.
DOWFAX 2A-1: sodium dodecylalkyl diphenyl ether disulfonate available from DOW
Pionine A-43S: sodium dodecyl diphenyl ether disulfonate available from Takemoto Oil and Fat Co., Ltd.
PELEX SS-L: sodium alkyl diphenyl ether disulfonate available from Kao Corporation
NEOPELEX No. 6: dodecylbenzene sulfonate available from Kao Corporation
ELEMINOL MON: sodium dodecylalkyl diphenyl ether disulfonate available from Sanyo Chemical Industries, Ltd.
Pionine A-43-N: ammonium dodecylalkyl diphenyl ether disulfonate available from Takemoto Oil and Fat Co., Ltd.

As is clear from Tables 8 to 10, provision of the first and second polishing steps enabled a lower scratch count to be achieved while reducing dishing as compared to a single stage polishing method.

It was revealed that the advantage of the present invention was particularly significant when the surfactant used was dodecyl diphenyl ether disulfonate, when the organic acid was an amino acid, and when the abrasive had a particle size of 20 to 40 nm.

We claim:

1. A metal polishing slurry comprising:
a compound represented by the general formula (1):
(X¹)ₙ-L General formula (1)
wherein X¹ represents a group having a heterocycle which contains at least one nitrogen atom, n represents an integer of 2 or more, and L represents a linking group having a valence of two or more, provided that X¹s whose number is n may be the same or different;
an oxidizer; and
an organic acid.

2. The metal polishing slurry according to claim 1 which is used in chemical mechanical polishing in a step of manufacturing semiconductor devices.

3. The metal polishing slurry according to claim 1, wherein the linking group represented by L in the general formula (1) contains at least one group selected from the group consisting of ureido group, thioureido group, amide group, ester group, carbonate group, carbamate group, sulfonamide group, sulfonureido group, hydroxy group, ether group, thioether group, amino group, carboxy group, sulfo group and heterocyclic group.

4. The metal polishing slurry according to claim 1, wherein the linking group represented by L in the general formula (1) contains at least one heterocycle.

5. The metal polishing slurry according to claim 4, wherein the heterocycle contained in the linking group represented by L in the general formula (1) is 1,2,4-triazole, benzotriazole or tetrazole.

6. The metal polishing slurry according to claim 1, wherein the heterocycle represented by X¹ in the general formula (1) is tetrazole, 1,2,4-triazole, 1,2,3-triazole or benzotriazole.

7. The metal polishing slurry according to claim 1, wherein the metal polishing slurry further contains a surfactant represented by the general formula (2):
R—Ar—O—Ar—SO₃⁻M⁺ General formula (2)
wherein R represents a linear or branched alkyl group having 8 to 20 carbon atoms, Ar represents an aryl group, and M⁺ represents hydrogen ion, an alkali metal ion or ammonium.

8. The metal polishing slurry according to claim 1 further containing colloidal silica.

9. The metal polishing slurry according to claim 8, wherein the colloidal silica has a primary particle size of 20 to 40 nm and an average association degree of up to 2.

10. The metal polishing slurry according to claim 8, wherein the colloidal silica is one in which at least some of surface silicon atoms are modified with aluminum atoms.

11. The metal polishing slurry according to claim 1, wherein the organic acid is an amino acid.

12. A method of chemical mechanical polishing comprising the steps of:
rotating a polishing platen on which a polishing pad is disposed;
feeding the metal polishing slurry of claim 1 to the polishing pad; and
polishing an object while moving the polishing pad and the object to be polished relative to each other with a surface of the object in contact with the polishing pad.

13. A compound represented by the general formula (3):
(X²)ₙ-L General formula (3)
wherein X² represents tetrazole, 1,2,4-triazole, 1,2,3-triazole or benzotriazole, L represents a linking group having a valence of 2 or more which contains at least one group selected from the group consisting of ureido group, thioureido group, amide group, ester group, sulfonamide group, sulfonureido group, hydroxy group, carbamate group, ether group, amino group, carboxy group, sulfo group and heterocyclic group, n is an inter of 2 or more; provided that X²s whose number is n may be the same or different.

14. The compound according to claim 13, wherein, in the general formula (3), X² represents tetrazole or 1,2,3-triazole, and the linking group having a valence of 2 or more as represented by L contains at least one group selected from the group consisting of ureido group, amide group, hydroxy group, carbamate group, ether group, and amino group.

15. The compound according to claim 13, wherein, in the general formula (3), n is 2 to 6, X² is tetrazole, and L contains at least one group selected from the group consisting of ureido group, amide group and carbamate group.
